(19) United States
(12) Patent Application Publication
(10) Pub. No.: US 2025/0143545 A1
(43) Pub. Date: May 8, 2025

(54) ENDOSCOPE PROTRUSION CALIBRATION

(71) Applicant: AURIS HEALTH, INC., Santa Clara, CA (US)

(72) Inventors: Michael Duckjune Kim, Dublin, CA (US); Chauncey F. Graetzel, Palo Alto, CA (US); Olivier Lavoie, Foster City, CA (US); Daniel Shalom Eliahu, Alamo, CA (US); Brandon Ritter, Stanford, CA (US)

(21) Appl. No.: 18/733,596

(22) Filed: Jun. 4, 2024

Related U.S. Application Data
(60) Provisional application No. 63/471,741, filed on Jun. 7, 2023.

(51) Int. Cl. A61B 1/00; G06T 7/00; G06T 7/11; G06T 7/246; G06T 7/50; G06T 7/60; G06T 7/80; G06T 7/90

(52) U.S. Cl. CPC A61B 1/00057; G06T 7/0012; G06T 7/11; G06T 7/246; G06T 7/50; G06T 7/60; G06T 7/80; G06T 7/90; G06T 2207/10024; G06T 2207/10068; G06T 2207/20081; G06T 2207/20084; G06T 2207/30061; G06T 2207/30244

(57) ABSTRACT

A robotic system capable of performing a protrusion calibration of an endoscope is disclosed herein. The endoscope includes an elongated scope with a sensor proximate a distal end and a tubular sheath, coaxially aligned with the elongated scope, which surrounds the elongated scope. The sheath and scope are movable relative to one another on a coaxial axis. The sensor may be a camera capable of capturing an opening formed by an inner lumen of the sheath positioned at a distal end of the sheath when the scope is retracted into the sheath such that the opening is made visible to the camera. A transition position where the sheath becomes visible from hidden may be detected based on analysis of readings from the sensor. Based on the transition position, distal ends of the sheath and the scope can be calibrated to provide a particular protrusion.

### CROSS-REFERENCE TO RELATED APPLICATION(S)

This application claims priority to U.S. Provisional Application No. 63/471,741, filed Jun. 7, 2023, entitled ENDOSCOPE PROTRUSION CALIBRATION, the disclosure of which is hereby incorporated by reference in its entirety.

### BACKGROUND

### Field

This disclosure relates to the field of medical devices. More particularly, the field pertains to systems and methods for robotic medical systems.

### Description of Related Art

Certain robotic medical procedures can involve the use of shaft-type medical instruments, such as endoscopes, which may be inserted into a subject through an opening (e.g., a natural orifice or a percutaneous access) and advanced to a target anatomical site. Such medical instruments can be articulatable, wherein the tip and/or other portion(s) of the shaft can deflect in one or more dimensions using robotic controls. An endoscope may include a scope coaxially aligned with and surrounded by a sheath.

### SUMMARY

A robotic system capable of performing a protrusion calibration of an endoscope is disclosed herein. The endoscope includes an elongated scope, with a sensor proximate the distal end of the scope, and a tubular sheath that is coaxially aligned with and covers the elongated scope. The sheath and scope are movable relative to each other on a coaxial axis. The robotic system includes at least one computer-readable memory in communication with at least one processor, the memory having stored thereon computer-executable instructions that when executed by the at one processor cause the at least one processor to determine a transition position based on data from the sensor. The sensor may be a camera capable of capturing images of an opening formed by an inner lumen of the sheath. The computer-executable instructions when executed by the at least one processor may further cause one or more actuators to cause relative movements of the scope and the sheath on the coaxial axis such that the sheath becomes visible to the camera. In one aspect, the scope is retracted into the sheath. The transition position may be where the sheath becomes visible to the camera. In one aspect, sensor data is filtered based on a color or other properties of the sheath and the transition position is determined based on filtered sensor data meeting a threshold. Based on the transition position, distal ends of the sheath and the scope can be calibrated to provide a particular protrusion distance, where protrusion is a relative position between the two distal ends. For example, a positive protrusion is when a scope distal end extends beyond a sheath distal end; a negative protrusion is when the sheath distal end extends beyond the scope distal end; and a zero protrusion is when the distal ends are aligned such that there is no protrusion.

In some aspects, the techniques described herein relate to a robotic system, including: an instrument including a scope and a sheath, the sheath aligned with the scope on a coaxial axis and surrounding the scope, the scope having a sensor proximate a distal end of the scope; and at least one computer-readable memory in communication with at least one processor, the memory having stored thereon computer-executable instructions that when executed cause the at least one processor to: calibrate a relative position of the distal end of the scope in relation to a distal end of the sheath based at least in part on a detection of the distal end of the sheath with sensor data captured with the sensor.

In some aspects, the techniques described herein relate to a robotic system, wherein the computer-executable instructions further cause the at least one processor to: execute a movement of the scope on the coaxial axis relative to the sheath; and wherein the detection is determined during the movement.

In some aspects, the techniques described herein relate to a robotic system, wherein the detection is determined during a retraction of the scope on the coaxial axis relative to the sheath.

In some aspects, the techniques described herein relate to a robotic system, wherein the calibration includes executing an extension of the scope on the coaxial axis after the detection to position the distal end of the scope at a standard protrusion in relation to the distal end of the sheath.

In some aspects, the techniques described herein relate to a robotic system, wherein the detection is determined based on a transition position, the transition position representing a position of the distal end of the scope relative to the distal end of the sheath whereby the at least one processor transitions between not detecting the sheath and detecting the sheath.

In some aspects, the techniques described herein relate to a robotic system, wherein the detection includes: filtering one or more images from the sensor that is a camera based on a color of the sheath; and determining that a filtered portion of the one or more images satisfies a threshold condition.

In some aspects, the techniques described herein relate to a robotic system, wherein determining that the filtered portion of the one or more images satisfies the threshold condition includes analyzing a single image.

In some aspects, the techniques described herein relate to a robotic system, wherein determining that the filtered portion of the one or more images satisfies the threshold condition includes analyzing multiple images.

In some aspects, the techniques described herein relate to a robotic system, wherein determining that the filtered portion of the one or more images satisfies the threshold condition includes comparing a pixel count of filtered portion remaining after the filtering to a threshold pixel count.

In some aspects, the techniques described herein relate to a robotic system, wherein determining that the filtered portion of the one or more images satisfies the threshold condition includes: detecting a geometrical shape in the filtered portion.

In some aspects, the techniques described herein relate to a robotic system, wherein determining that the filtered portion of the one or more images satisfies the threshold further includes: determining a center position of the geometrical shape that is circular; and determining that the center position is within a range of variance.

In some aspects, the techniques described herein relate to a robotic system, wherein the computer-executable instructions further cause the at least one processor to: maintain an alignment between the scope and the sheath on a coaxial axis based on the relative position.

In some aspects, the techniques described herein relate to a system for calibrating an endoscope, the system including: a scope; a camera proximate a distal end of the scope; a sheath surrounding and coaxially aligned with the scope; and at least one computer-readable memory in communication with at least one processor, the memory having stored thereon computer-executable instructions that, when executed, cause the at least one processor to: determine a transition position representing a position of a distal end of the scope relative to a distal end of the sheath where the sheath becomes detectable in an image captured by the camera; and cause a coaxial movement of the scope relative to the sheath based at least in part on the transition position and an offset.

In some aspects, the techniques described herein relate to a system, wherein the first image and the second image are captured during a change in the position of the distal end of the scope relative to the distal end of the sheath.

In some aspects, the techniques described herein relate to a system, wherein the determining the transition position includes: filtering the second image based on a color of the sheath; determining that a filtered portion of the second image satisfies a threshold condition; and in response to the determination that the filtered portion satisfies the threshold condition, determining that a sheath is detected.

In some aspects, the techniques described herein relate to a system, wherein the determining the transition position includes: generating a binary image based on the filtered portion.

In some aspects, the techniques described herein relate to a system, wherein the determining that the filtered portion of the second image satisfies the threshold condition includes: masking the filtered portion with an inverse shape mask.

In some aspects, the techniques described herein relate to a system, wherein the determining that the filtered portion of the second image satisfies the threshold condition includes: applying the inverse shape mask to the filtered portion to generate a masked image; and counting pixels in each quadrant of the masked image.

In some aspects, the techniques described herein relate to a system, wherein the determining that the filtered portion of the second image satisfies the threshold condition includes: masking the filtered portion with a segmentation mask generated using a trained neural network.

In some aspects, the techniques described herein relate to a method for calibrating a protrusion of a scope relative to a sheath that surrounds and is coaxially aligned with the scope, the method including: capturing one or more images with a camera proximate a distal end of the scope; filtering the one or more images based on a visual property of the sheath to generate a filtered portion; determining that the filtered portion satisfies a threshold; determining a transition position; and determining a target protrusion based at least in part on the transition position.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example medical system, in accordance with some implementations.

FIG. 2 illustrates a schematic view of the example medical system of FIG. 1, in accordance with some implementations.

FIG. 3 illustrates an example robotically controllable sheath and scope assembly, in accordance with one or more implementations.

FIG. 4 is an illustration of an example robotic system that is capable of controlling protrusion of a coaxially aligned scope and sheath pair, in accordance with some implementations.

FIG. 5 illustrates an example system including a protrusion calibration framework, in accordance with some implementations.

FIG. 6 is a flow diagram of an example process for calibrating protrusion of a scope and sheath combination, in accordance with some implementations.

FIG. 7 is a set of illustrations of cross sections of the scope and sheath pair during transition position determination, in accordance with some implementations.

FIG. 8 is a set of illustrations of cross sections of the scope and sheath pair at a distal portion of the endoscope during calibration, in accordance with some implementations.

FIGS. 9A-9B are illustrations of a scope and sheath pair at pre-calibration and post-calibration, in accordance with some implementations.

FIG. 10 illustrates an example process for detecting an inner lumen of a sheath from an image, in accordance with some implementations.

FIG. 11 is an example diagram showing various filters and/or masks applied to images to extract various information used for sheath detection, in accordance with some implementations.

FIG. 12 is an example block diagram of a sheath detection system, in accordance with some implementations.

FIG. 13 is an example flow diagram of a calibration decision process involving multiple approaches, in accordance with some implementations.

FIG. 14 is a set of images showing a single-frame approach, in accordance with some implementations.

FIG. 15 is a set of images showing a multi-frame approach, in accordance with some implementations.

FIG. 16 is a set of images showing a detected sheath image, no detection image, and an insufficient detection image, in accordance with some implementations.

FIG. 17 is an example user interface for protrusion calibration, in accordance with some implementations.

FIG. 18 is a schematic of a computer system that may be implemented by the control system, robotic system, or any other component or module in the disclosed subject matter that performs computations, stores data, processes data, and executes instructions, in accordance with some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts, features associated with separate figures that are identified by common reference numbers are not related and/or similar with respect to at least certain aspects.

### Overview

The present disclosure relates to systems, devices, and methods for calibrating a shaft-type medical instrument, such as an endoscope. Some shaft-type medical instruments include multiple coaxially aligned shafts that are configured to move in relation to one another. For example, an endoscope may comprise a scope surrounded by a sheath where both the scope and the sheath can be independently extended or retracted in relation to each other. The scope may be an internal shaft configured to slide within a tube-like outer shaft. Optimizing relative position of the inner shaft and the outer shaft of the shaft-type medical instrument can improve system performance.

With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.” Furthermore, the term “shaft” is used herein according to its broad and ordinary meaning and may refer to any type of elongate cylinder, tube, scope, prism (e.g., rectangular, oval, elliptical, or oblong prism), wire, or similar, regardless of cross-sectional shape. It should be understood that any reference herein to a “shaft” or “instrument shaft” can be understood to possibly refer to an endoscope. The term “endoscope” is used herein according to its broad and ordinary meaning, and may refer to any type of elongate (e.g., shaft-type) medical instrument having image generating, viewing, and/or capturing functionality and being configured to be introduced into any type of organ, cavity, lumen, chamber, or space of a body. Endoscopes, in some instances, may comprise an at least partially rigid and/or flexible tube, and may be dimensioned to be passed within an outer sheath, catheter, introducer, or other lumen-type device, or may be used without such devices. The term “scope” herein may refer to the shaft portion of an endoscope that is positioned inside of a sheath that is coaxially aligned to the scope. For convenience in description, the inner shaft will be referred as the scope and the outer shaft will be referred as the sheath but it will be understood that additional coaxial shafts may be layered internal to the scope or external to the sheath.

A gap formed between a distal end of the scope and a distal end of the sheath may be referred as a “protrusion.” The protrusion may be measured and provided as a distance metric, for example, in millimeters (mm). For a given medical procedure, such as a bronchoscopy, there may be a desirable protrusion for a scope and sheath pair of an endoscope that may facilitate the medical procedure. For instance, it may be advantageous to maintain a 5 mm protrusion before entry or during navigation to a site. As will be described in greater detail, a calibration of the endoscope can help maintain or otherwise provide the desired protrusion (e.g., a target protrusion).

A calibration procedure may involve moving (extending or retracting) the scope relative to the sheath, the sheath relative to the scope, or a combination thereof. A camera or sensor proximate a distal end of the scope may capture images (e.g. provide image data) during the movement. The images may depict an outline of an inner sheath opening at a distal end of the sheath. When the scope retracts in relation to the sheath, the captured images may reflect the opening transitioning from not visible (hidden) to visible. Conversely, when the scope extends in relation to the shaft, the captured images may reflect the opening transitioning from visible to not visible (hidden). In some examples, image processing may detect whether there is a transition of the sheath opening from hidden to visible and vice versa, and log a transition position when the transition has been detected.

The scope and sheath pair can be set to a target protrusion based on the transition position and expected change in protrusion based on a kinematic model. For example, a robotic system may log robot data and/or kinematic data of the sheath and the scope at the transition position, determine an amount to further extend/retract the scope/sheath based on a kinematic model used to control the scope/sheath such that the pair can provide the target protrusion from the transition position, and extend/retract the scope/sheath by the determined amount.

### Example Medical System

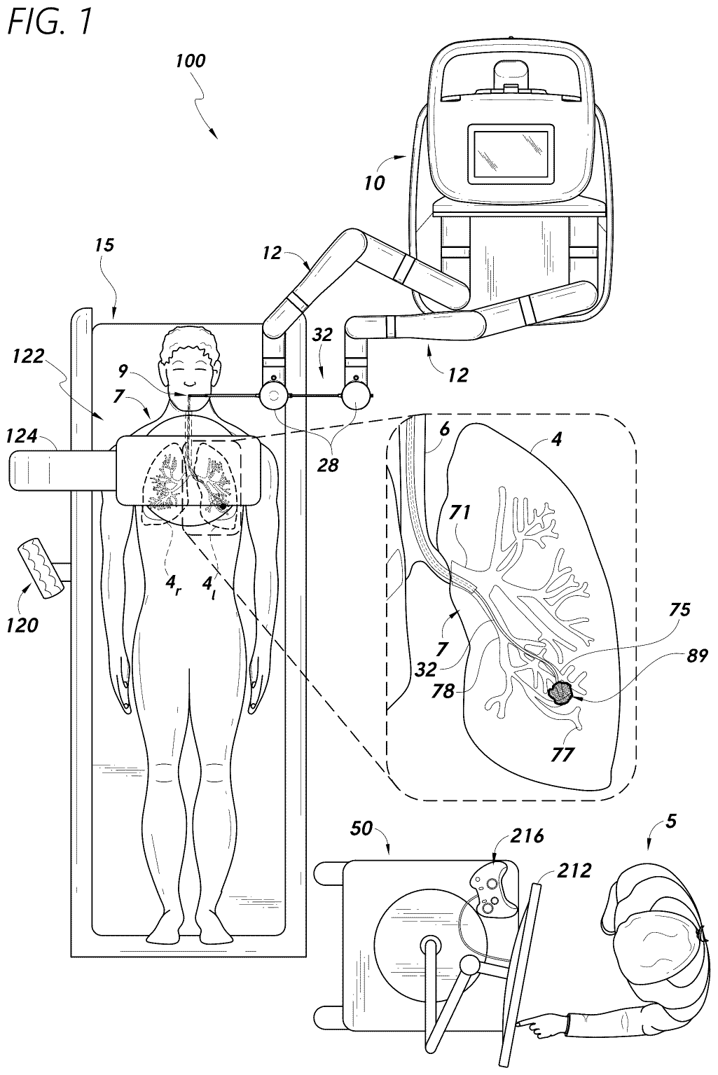

FIG. 1 illustrates an example medical system 100 (also referred to as “surgical medical system 100” or “robotic medical system 100”) in accordance with one or more examples. For example, the medical system 100 can be arranged for diagnostic and/or therapeutic bronchoscopy, as shown. The medical system 100 can include and utilize a robotic system 10, which can be implemented as a robotic cart, for example. Although the medical system 100 is shown as including various cart-based systems/devices, the concepts disclosed herein can be implemented in any type of robotic system/arrangement, such as robotic systems employing rail-based components, table-based robotic end-effectors/robotic manipulators, etc. The robotic system 10 can comprise one or more robotic arms 12 (also referred to as “robotic positioner(s)”) configured to position or otherwise manipulate a medical instrument, such as a medical instrument 32 (e.g., a steerable endoscope or another elongate instrument having a flexible elongated body). For example, the medical instrument 32 can be advanced through a natural orifice access point (e.g., the mouth 9 of a subject 7, positioned on a table 15 in the present example) to deliver diagnostic and/or therapeutic treatment. Although described in the context of a bronchoscopy procedure, the medical system 100 can be implemented for other types of procedures, such as gastro-intestinal (GI) procedures, renal/urological/nephrological procedures, etc. The term “subject” is used herein to refer to live patient and human anatomy as well as any subjects to which the present disclosure may be applicable. For example, the “subject” may refer to subjects including physical anatomic models (e.g., anatomical education model, anatomical model, medical education anatomy model, etc.) used in dry runs, models in computer simulations, or the like that covers non-live patients or test subjects.

With the robotic system 10 properly positioned, the medical instrument 32 can be inserted into the subject 7 robotically, manually, or a combination thereof. In examples, the one or more robotic arms 12 and/or instrument driver(s) 28 thereof can control the medical instrument 32. The instrument driver(s) 28 can be repositionable in space by manipulating the one or more robotic arms 12 into different angles and/or positions.

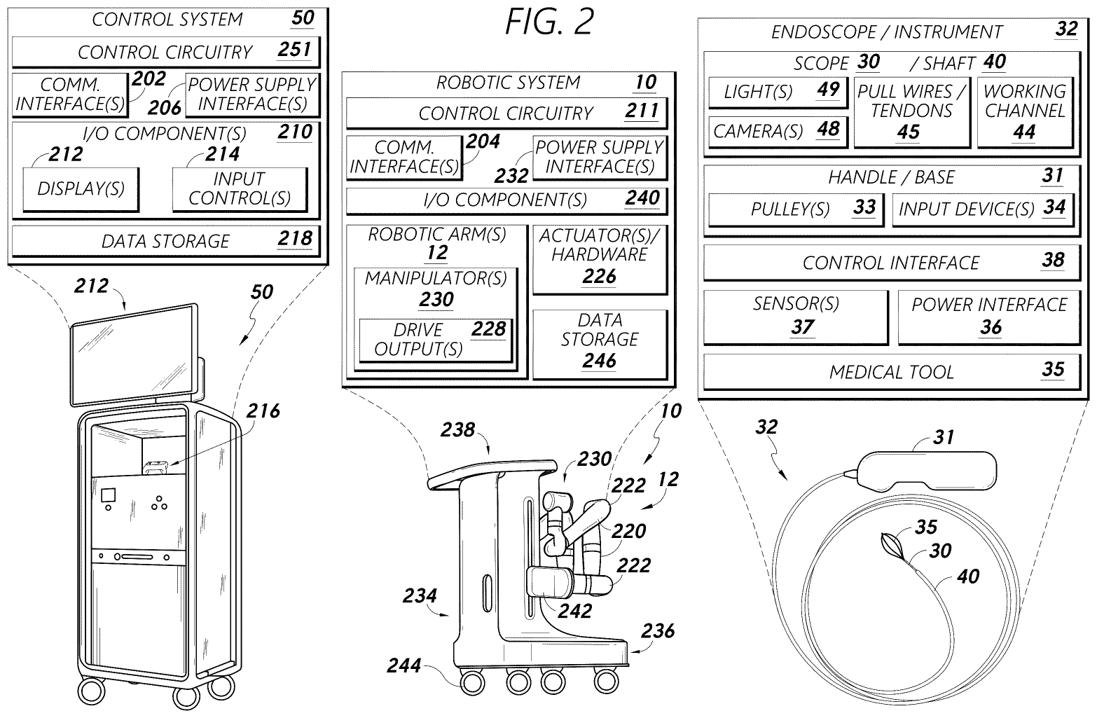

The medical system 100 can also include a control system 50 (also referred to as “control tower” or “mobile tower”), described in detail below with respect to FIG. 2. The control system 50 can include one or more displays 212 to provide/display/present various information related to medical procedures, such as anatomical images. The control system 50 can additionally include one or more control mechanisms, which may be a separate directional input control 216 or a graphical user interface (GUI) presented on the displays 212.

In some examples, the display 212 can be a touch-capable display, as shown, that may present anatomical images and allow selection thereon. Few example anatomical images can include CT images, fluoroscopic images, images of an anatomical map, or the like. With the touch-capable display, an operator 5 reviewing the images may find it convenient to identify targets (e.g., target objects or a target region of interest) within the images using a touch-based selection instead of using the directional input control 216. For example, the operator 5 may select a scope tip and/or a nodule using a touchscreen.

The control system 50 can be communicatively coupled (e.g., via wired and/or wireless connection(s)) to the robotic system 10 to provide support for controls, electronics, fluidics, optics, sensors, and/or power to the robotic system 10. Placing such functionality in the control system 50 can allow for a smaller form factor of the robotic system 10 that may be more easily adjusted and/or re-positioned by an operator 5. Additionally, the division of functionality between the robotic system 10 and the control system 50 can reduce operating room clutter and/or facilitate efficient clinical workflow.

The medical system 100 can include an electromagnetic (EM) field generator 120, which is configured to broadcast/emit an EM field that is detected by EM sensors, such as a sensor associated with the medical instrument 32. The EM field can induce small currents in coils of EM sensors (also referred to as “position sensors”), which can be analyzed to determine a pose (position and/or angle/orientation) of the EM sensors relative to the EM field generator 120. In some examples, the EM sensors may be positioned at a distal end of the medical instrument 32 and a pose of the distal end may be determined in connection with the pose of the EM sensors. Although EM fields and EM sensors are described in many examples herein, position sensing systems and/or sensors can be any type of position sensing systems and/or sensors, such as optical position sensing systems/sensors, image-based position sensing systems/sensors, etc.

The medical system 100 can further include an imaging system 122 (e.g., a fluoroscopic imaging system) configured to generate and/or provide/send image data (also referred to as “image(s)”) to another device/system. For example, the imaging system 122 can generate image data depicting anatomy of the subject 7 and provide the image data to the control system 50, robotic system 10, a network server, a cloud server, and/or another device. The imaging system 122 can comprise an emitter/energy source (e.g., X-ray source, ultrasound source, or the like) and/or detector (e.g., X-ray detector, ultrasound detector, or the like) integrated into a supporting structure (e.g., mounted on a C-shaped arm support 124), which may provide flexibility in positioning around the subject 7 to capture images from various angles without moving the subject 7. Use of the imaging system 122 can provide visualization of internal structures/anatomy, which can be used for a variety of purposes, such as navigation of the medical instrument 32 (e.g., providing images of internal anatomy to the operator 5), localization of the medical instrument 32 (e.g., based on an analysis of image data), etc. In examples, use of the imaging system 122 can enhance the efficacy and/or safety of a medical procedure, such as a bronchoscopy, by providing clear, continuous visual feedback to the operator 5.

In some examples, the imaging system 122 is a mobile device configured to move around within an environment. For instance, the imaging system 122 can be positioned next to the subject 7 (as illustrated) during a particular phase of a procedure and removed when the imaging system 122 is no longer needed. In other examples, the imaging system 122 can be part of the table 15 or other equipment in an operating environment. The imaging system(s) 122 can be implemented as a Computed Tomography (CT) machine/system, X-ray machine/system, fluoroscopy machine/system, Positron Emission Tomography (PET) machine/system, PET-CT machine/system, CT angiography machine/system, Cone-Beam CT (CBCT) machine/system, 3DRA machine/system, single-photon emission computed tomography (SPECT) machine/system, Magnetic Resonance Imaging (MRI) machine/system, Optical Coherence Tomography (OCT) machine/system, ultrasound machine/system, etc. In some cases, the medical system 100 includes multiple imaging system, such as a first type of imaging system and a second type of imaging system, wherein the different types of imaging systems can be used or positioned over the subject 7 during different phases/portions of a procedure depending on the needs at that time.

In some examples, the imaging system 122 can be configured to generate a three-dimensional (3D) model of an anatomy. For example, the imaging system 122 is configured to process multiple images (also referred to as “image data,” in some cases) to generate the 3D model. For example, the imaging system 122 can be implemented as a CT machine configured to capture/generate a series of images/image data (e.g., 2D images/slices) from different angles around the subject 7, and then use one or more algorithms to reconstruct these images/image data into a 3D model. The 3D model can be provided to the control system 50, robotic system 10, a network server, a cloud server, and/or another device, such as for processing, display, or otherwise.

In the interest of facilitating descriptions of the present disclosure, FIG. 1 illustrates a respiratory system as an example anatomy. The respiratory system includes the upper respiratory tract, which comprises the nose/nasal cavity, the pharynx (i.e., throat), and the larynx (i.e., voice box). The respiratory system further includes the lower respiratory tract, which comprises the trachea 6, the lungs 4 (4r and 4l), and the various segments of the bronchial tree. The bronchial tree includes primary bronchi 71, which branch off into smaller secondary 78 and tertiary 75 bronchi, and terminate in even smaller tubes called bronchioles 77. Each bronchiole tube is coupled to a cluster of aveoli (not shown). During the inspiration phase of the respiratory cycle, air enters through the mouth and nose and travel down the throat into the trachea 6, into the lungs 4 through the right and left main bronchi 71, into the smaller bronchi airways 78, 75, into the smaller bronchiole tubes 77, and into the alveoli, where oxygen and carbon dioxide exchange takes place.

The bronchial tree is an example luminal network in which robotically-controlled instruments may be navigated and utilized in accordance with the inventive solutions presented here. However, although aspects of the present disclosure are presented in the context of luminal networks including a bronchial network of airways (e.g., lumens, branches) of a subject's lung, some examples of the present disclosure can be implemented in other types of luminal networks, such as renal networks, cardiovascular networks (e.g., arteries and veins), gastrointestinal tracts, urinary tracts, etc.

FIG. 2 illustrates example components of the control system 50, robotic system 10, and medical instrument 32, in accordance with one or more examples. The control system 50 can be coupled to the robotic system 10 and operate in cooperation therewith to perform a medical procedure. For example, the control system 50 can include communication interface(s) 202 for communicating with communication interface(s) 204 of the robotic system 10 via a wireless or wired connection (e.g., to control the robotic system 10). Further, in examples, the control system 50 can communicate with the robotic system 10 to receive position/sensor data therefrom relating to the position of sensors associated with an instrument/member controlled by the robotic system 10. In some examples, the control system 50 can communicate with the EM field generator 120 to control generation of an EM field in an area around a subject 7. The control system 50 can further include a power supply interface(s) 206.

The control system 50 can include control circuitry 251 configured to cause one or more components of the medical system 100 to actuate and/or otherwise control any of the various system components, such as carriages, mounts, arms/positioners, medical instruments, imaging devices, position sensing devices, sensor, etc. Further, the control circuitry 251 can be configured to perform other functions, such as cause display of information, process data, receive input, communicate with other components/devices, and/or any other function/operation discussed herein.

The control system 50 can further include one or more input/out (I/O) components 210 configured to assist a physician or others in performing a medical procedure. For example, the one or more I/O components 210 can be configured to receive input and/or provide output to enable a user to control/navigate the medical instrument 32, the robotic system 10, and/or other instruments/devices associated with the medical system 100. The control system 50 can include one or more displays 212 to provide/display/present various information regarding a procedure. For example, the one or more displays 212 can be used to present navigation information including a virtual anatomical model of anatomy with a virtual representation of a medical instrument, image data, and/or other information. The one or more I/O components 210 can include a user input control(s) 214, which can include any type of user input (and/or output) devices or device interfaces, such as a directional input control(s) 216, touch-based input control(s) including gesture-based input control(s), motion-based input control(s), or the like. The user input control(s) 214 may include one or more buttons, keys, joysticks, handheld controllers (e.g., video-game-type controllers), computer mice, trackpads, trackballs, control pads, sensors (e.g., motion sensors or cameras) that capture hand gestures and finger gestures, touchscreens, toggle (e.g., button) inputs, and/or interfaces/connectors therefore. In examples, such input(s) can be used to generate commands for controlling medical instrument(s), robotic arm(s), and/or other components.

The control system 50 can also include data storage 218 configured to store executable instruments (e.g., computer-executable instructions) that are executable by the control circuitry 251 to cause the control circuitry 251 to perform various operations/functionality discussed herein. In examples, two or more of the components of the control system 50 can be electrically and/or communicatively coupled to each other.

The robotic system 10 can include the one or more robotic arms 12 configured to engage with and/or control, for example, the medical instrument 32 and/or other elements/components to perform one or more aspects of a procedure. As shown, each robotic arm 12 can include multiple segments 220 coupled to joints 222, which can provide multiple degrees of movement/freedom. The number of segments 220 and/or the joints 222 may be determined based on a desired degrees of freedom. For example, where seven degrees of freedom is desired, the number of joints 222 can be seven or more where additional joints can provide redundant degree of freedom.

The robotic system 10 can be configured to receive control signals from the control system 50 to perform certain operations, such as to position one or more of the robotic arms 12 in a particular manner, manipulate an instrument, and so on. In response, the robotic system 10 can control, using control circuitry 211 thereof, actuators 226 and/or other components of the robotic system 10 to perform the operations. For example, the control circuitry 211 can control insertion/retraction, articulation, roll, etc. of a shaft of the medical instrument 32 or another instrument by actuating a drive output(s) 228 of a robotic manipulator(s) 230 (e.g., end-effectors) coupled to a base of a robotically-controllable instrument. The drive output(s) 228 can be coupled to a drive input on an associated instrument, such as an instrument base of an instrument that is coupled to the associated robotic arm 12. The robotic system 10 can include one or more power supply interfaces 232.

The robotic system 10 can include a support column 234, a base 236, and/or a console 238. The console 238 can provide one or more I/O components 240, such as a user interface for receiving user input and/or a display screen (or a dual-purpose device, such as a touchscreen) to provide the physician/user with preoperative and/or intraoperative data. The support column 234 can include an arm support 242 (also referred to as “carriage”) for supporting the deployment of the one or more robotic arms 12. The arm support 242 can be configured to vertically translate along the support column 234. Vertical translation of the arm support 242 allows the robotic system 10 to adjust the reach of the robotic arms 12 to meet a variety of table heights, subject sizes, and/or physician preferences. The base 236 can include wheel-shaped casters 244 (also referred to as “wheels”) that allow for the robotic system 10 to move around the operating room prior to a procedure. After reaching the appropriate position, the casters 244 can be immobilized using wheel locks to hold the robotic system 10 in place during the procedure.

The joints 222 of each robotic arm 12 can each be independently controllable and/or provide an independent degree of freedom available for instrument navigation. For example, each actuator 226 can individually control a joint 222 without affecting control of other joints 222 and each joint 222 can individually move without causing movements of other joints 222. Similarly, each robotic arm 12 can be individually controlled without affecting movement of other robotic arms 12. The independently controlled actuators 226, joints 222, and arms 12 may be controlled in a coordinated manner to provide complex movements.

In some examples, each robotic arm 12 has seven joints, and thus provides seven degrees of freedom, including “redundant” degrees of freedom. Redundant degrees of freedom can allow robotic arms 12 to be controlled to position their respective robotic manipulators 230 at a specific position, orientation, and/or trajectory in space using different linkage positions and joint angles. This allows for the robotic system 10 to position and/or direct a medical instrument from a desired point in space while allowing the physician to move the joints 222 into a clinically advantageous position away from the patient to create greater access, while avoiding collisions.

The one or more robotic manipulators 230 (e.g., end-effectors) can be couplable to an instrument base/handle, which can be attached using a sterile adapter component in some instances. The combination of the robotic manipulator 230 and coupled instrument base, as well as any intervening mechanics or couplings (e.g., sterile adapter), can be referred to as a robotic manipulator assembly, or simply a robotic manipulator. Robotic manipulator/robotic manipulator assemblies can provide power and/or control interfaces. For example, interfaces can include connectors to transfer pneumatic pressure, electrical power, electrical signals, and/or optical signals from the robotic arm 12 to a coupled instrument base. Robotic manipulator/robotic manipulator assemblies can be configured to manipulate medical instruments (e.g., surgical tools/instruments) using techniques including, for example, direct drives, harmonic drives, geared drives, belts and/or pulleys, magnetic drives, and the like.

The robotic system 10 can also include data storage 246 configured to store executable instruments (e.g., computer-executable instructions) that are executable by the control circuitry 211 to cause the control circuitry 211 to perform various operations/functionality discussed herein. In example, two or more of the components of the robotic system 10 can be electrically and/or communicatively coupled to each other.

Data storage (including the data storage 218, data storage 246, and/or other data storage/memory) can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Control circuitry (including the control circuitry 251, control circuitry 211, and/or other control circuitry) can include circuitry embodied in a robotic system, control system/tower, instrument, or any other component/device. Control circuitry can include any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein can further include one or more circuit substrates (e.g., printed circuit boards), conductive traces and vias, and/or mounting pads, connectors, and/or components. Control circuitry can further comprise one or more storage devices, which may be embodied in a single device, a plurality of devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. In examples in which control circuitry comprises a hardware and/or software state machine, analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Functionality described herein can be implemented by the control circuitry 251 of the control system 50 and/or the control circuitry 211 of the robotic system 10, such as by the control circuitry 251, 211 executing executable instructions to cause the control circuitry 251, 211 to perform the functionality.

The endoscope/medical instrument 32 includes a handle or base 31 coupled to an endoscope shaft. For example, the endoscope (also referred herein as “shaft”) can include the elongate shaft including one or more lights 49 and one or more cameras 48 or other imaging devices. The camera 48 can be a part of the scope 30 or can be a separate camera assembly that may be introduced into a working channel 44.

The medical instrument 32 can be powered through a power interface 36 and/or controlled through a control interface 38, each or both of which may interface with a robotic arm/component of the robotic system 10. The medical instrument 32 may further comprise one or more sensors 37, such as pressure sensors and/or other force-reading sensors, which may be configured to generate signals indicating forces experienced at/by one or more components of the medical instrument 32.

The medical instrument 32 can include coaxially aligned shafts-type instruments that are independently controllable. In some examples, a first shaft-type instrument can be a scope 30 and a second shaft-type instrument can be a sheath 40. The scope 30 may be slidably positioned within a working channel/lumen of the sheath 40. The terms “lumen” and “channel” are used herein according to their broad and ordinary meanings and may refer to a physical structure forming a cavity, void, conduit, or other pathway, such as an at least partially rigid elongate tubular structure, or may refer to a cavity, void, pathway, or other channel, itself, that occupies a space within an elongate structure (e.g., a tubular structure). The telescopic arrangement of the scope 30 and the sheath 40 may allow for a relatively thin design of the scope 30 and may improve a bend radius of the scope 30 while providing a structural support via the sheath 40.

The medical instrument 32 includes certain mechanisms for causing the scope 30 and/or sheath 40 to articulate/deflect with respect to an axis thereof. For example, the scope 30 and/or sheath 40 may have been associated with a proximal portion thereof, one or more drive inputs 34 associated, and/or integrated with one or more pulleys/spools 33 that are configured to tension/untension pull wires/tendons 45 of the scope 30 and/or sheath 40 to cause articulation of the scope 30 and/or sheath 40. Articulation of one or both of the scope 30 and/or sheath 40 may be controlled robotically, such as through operation of robotic manipulators 230 associated with the robot arm(s) 12, wherein such operation may be controlled by the control system 11 and/or robotic system 10.

The scope 30 can further include one or more working channels 44, which may be formed inside the elongate shaft and run a length of the scope 30. The working channel 44 may serve for deploying therein a medical tool 35 or a component of the medical instrument 32 (e.g., a lithotripter, a basket 35, forceps, laser, camera 48, or the like) or for performing irrigation and/or aspiration, out through a scope distal end 430, into an operative region surrounding the distal end. The medical instrument 32 may be used in conjunction with a medical tool 35 and include various hardware and control components for the medical tool 35 and, in some instances, include the medical tool 35 as part of the medical instrument 32. For example, as shown, the medical instrument 32 can comprise a basket formed of one or more wire tines but any medical tool 35 are contemplated.

### Robotic Scope/Sheath Navigation

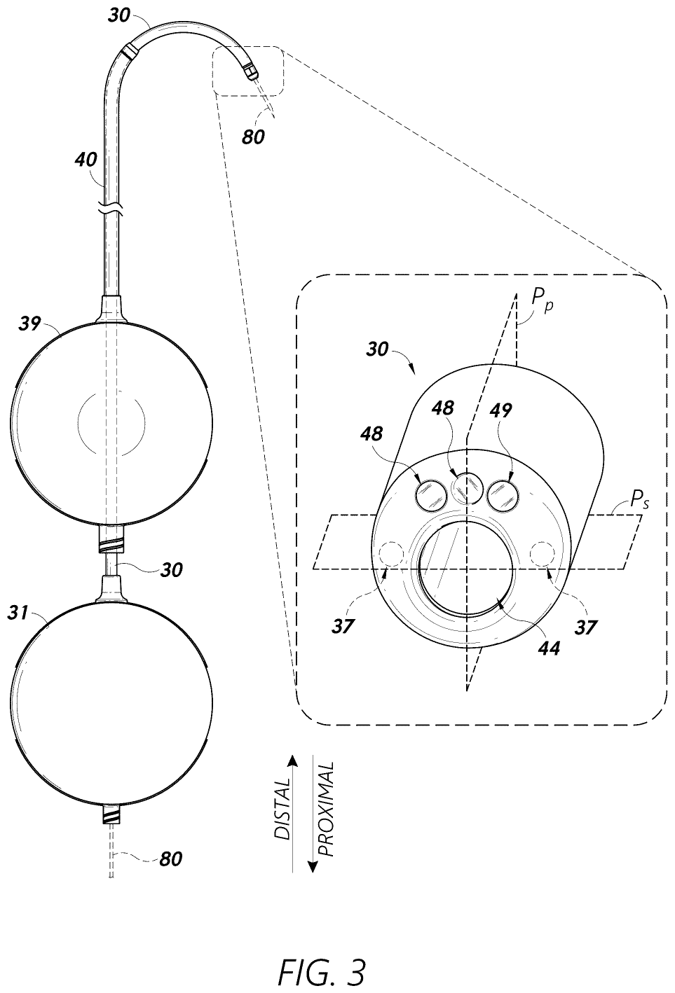

FIG. 3 illustrates an example robotically controllable sheath 40 and scope 30 assembly, in accordance with some implementations. The scope 30 can include a base 31 configured to be coupled to a robotic manipulator (e.g., the robotic manipulator 230 of FIG. 2) to facilitate robotic control/advancement of the scope 30. Another robotic manipulator may be coupled to a base 39 associated with the sheath 40 to facilitate advancement and/or articulation of the sheath 40. It should be understood that the scope 30 and sheath 40 shown in FIG. 3 and described in connection therewith can be any type of medical instrument, such as any type of steerable sheath or catheter that may be utilized in connection with procedures/processes disclosed herein.

FIG. 3 includes a detailed image of a distal portion of the assembly. The scope 30 may include one or more working channels 44 through which additional instruments/tools (e.g., the medical tool 35), such as injection and/or biopsy needles, lithotripters, basketing devices, forceps, or the like, can be introduced into a treatment site. The scope 30 can be inserted through the lumen of the sheath 40 such that the scope 30 and/or sheath 40 can be controlled in a telescoping manner based on commands received from a user and/or automatically generated by a robotic system. In some implementations, a working channel instrument 80 (e.g., biopsy needle) may be coupled to a robotic manipulator, disposed within a working channel of the scope 30, and/or controlled in concert with the other instruments.

Each of the robotically controllable instruments may be articulable with a number of degrees of freedom. For example, an endoscope may be configured to move/articulate in multiple degrees of freedom, such as: insertion, roll, and articulation in various directions. In implementations in which an endoscope is manipulated within a controllable outer access sheath, the system may provide up to ten degrees of freedom, or more (e.g., for each instrument, the degrees of freedom may include: one insertion degree of freedom and four (or more) independent pull wires, each providing an independent articulation degree of freedom), which can allow for compound bending of the instrument.

Robotically controllable endoscopes in accordance with the present disclosure can be configured to provide relatively precise control near the distal tip/portion of the endoscope, which can be advantageous particularly after the endoscope has already been significantly bent or deflected to reach the desired target. The scope 30 and/or sheath 40 can be deflectable in one or two directions in each of two planes (e.g., Pₚ, Pₛ). One or more articulation control pull wires, which may have the form of any type of elongate cable, wire, tendon, or the like, can run along the outer surface of, and/or at least partially within, the shaft of the scope 30 and/or sheath 40. Any reference herein to a pull wire may be understood to refer to any segment of a pull wire. That is, description herein of pull wires can be understood to refer more generally to pull wire segments, which may comprise an entire wire end-to-end, or any length or subsegment thereof. The one or more pull wires of an articulable instrument described herein can include one, two, three, four, five, six or more pull wires or segments. Manipulation of the one or more pull wires can produce articulation of the articulation section of the associated instrument. Manipulation of the one or more pull wires can be controlled via one or more instrument drivers positioned within, or connected to, the instrument base. For example, the robotic attachment interface between the instrument base and the robotic manipulator can include one or more mechanical inputs (e.g., receptacles, pulleys, gears, spools), that are designed to be reciprocally mated with one or more torque couplers on an attachment surface of the robotic manipulator. Drive inputs associated with the instrument base can be configured to control or apply tension to the plurality of pull wires in response to drive outputs from the robotic manipulator. The pull wires may include any suitable or desirable materials, including any metallic and non-metallic materials such as stainless steel, Kevlar, tungsten, carbon fiber, and the like.

FIG. 3 shows the scope 30 positioned within the inner channel of the sheath 40. In some implementations, the scope 30 and the sheath 40 are independently controlled relative to one another. For example, a robotic manipulator coupled to the endoscope base 31 can move the endoscope base 31 to insert or retract the scope 30 relative to the sheath 40 and/or patient anatomy. Similarly, the robotic manipulator coupled to the sheath base 39 can move the sheath base 39 to insert or retract the sheath 40 relative to the scope 30 and/or patient anatomy. In some embodiments, only distal portions of the scope 30 and/or sheath 40 are articulable.

### Measuring Protrusion

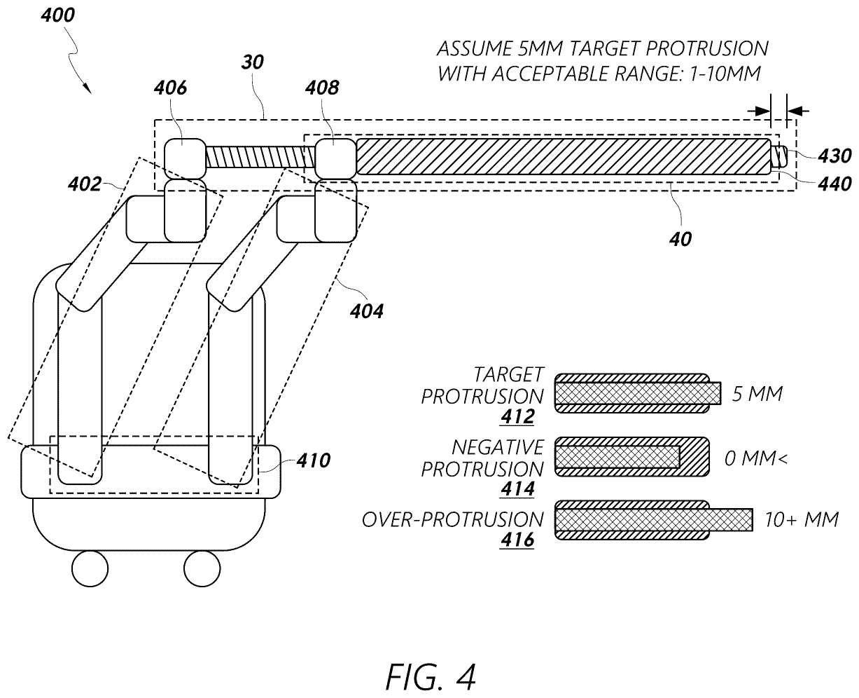

FIG. 4 is an illustration of an example robotic system 400 that is capable of controlling protrusion of a coaxially aligned scope and sheath pair, in accordance with some implementations. The example robotic system 400 can be a combination of the robotic system 10 and the medical instrument 32 (e.g., endoscope) comprising the scope 30 and the sheath 40 in FIG. 2. The example robotic system depicts a first control assembly 402 and a second control assembly 404 coupled to and supported by a robotic system base 410.

As shown in FIG. 4, the first control assembly 402 is coupled to the scope 30 and the second control assembly 404 is coupled to the sheath 40 coaxially surrounding (e.g., covering) the scope 30. Each of the first control assembly 402 and the second control assembly 404 may comprise a robotic arm (e.g., the robotic arm 12 of FIG. 2) having a series of linking arm segments (e.g., the segments 220 of FIG. 2) that are connected by a series of joints (e.g., the joints 222 of FIG. 2) and terminate at a distal end with a robotic manipulator (e.g., the robotic manipulators 230 of FIG. 2). Each of the robotic manipulators is configured to extend or retract a shaft coupled to the robotic manipulator.

In FIG. 4, a proximal end of the sheath 40 is coupled to a first robotic manipulator 406 of the first control assembly 402 and a proximal end of the scope 30 is coupled to a second robotic manipulator 408 of the second control assembly 404. Thus, the scope 30 and the sheath 40 may be articulated and/or moved independently through isolated/independent operation of the first robotic manipulator 406 and the second robotic manipulator 408, thereby providing independent extension or retraction of the scope 30 and the sheath 40.

The term “extension” may refer to an action that causes a length of a shaft (e.g., the scope, the sheath, or the endoscope) to increase as measured from where the shaft is coupled to a component (e.g., manipulator, drive output, end effector, robotic arm, etc.) controlling the length. When the length of the shaft is not curved, the extension will position a distal end of the shaft to be further away from the component. When the length of the shaft has a U-turn (a greater than 90 degrees deflection), it is possible that the extension will position the distal end of the shaft closer to the component. The scope 30 in FIG. 3 is positioned with an example U-turn. Here, for ease of descriptions, it will be assumed that the shaft is not articulated beyond 90 degrees and extension will result in insertion or advancement. Conversely, the term “retraction” may refer to an action that causes the length of the shaft to decrease as measured from the component. For ease of descriptions, it will be assumed that the shaft is not articulated beyond 90 degrees and retraction will result in contraction or retreat.

As briefly described before, a “protrusion” is a distance metric that can measure the difference between a scope distal end 430 and a sheath distal end 440, which may change based on extension and retraction of either of the scope distal end 430 and the sheath distal end 440. Protrusion may be measured as a distance metric of the position of the scope distal end 430 subtracted by a distance metric of the position of the sheath distal end 440, where the distance metrics of the positions are measured from a common reference point proximate the robotic system. Alternatively, protrusion may be measured as a distance metric of how much further the sheath distal end 440 extends beyond the scope distal end 430. As measured, a protrusion may refer to both a positive protrusion (greater than zero protrusion) where the scope distal end 430 extends beyond the sheath distal end 440 and a negative protrusion (less than zero protrusion) where the sheath distal end 440 extends beyond the scope distal end 430.

In some instances, it may be advantageous to control protrusion. For example, the scope 30 and the sheath 40 can be driven to achieve a target protrusion such that the sheath 40 can protect and provide support and protection for the scope 30 while camera proximate the scope distal end 430 can provide visual feedback without obstruction from the sheath distal end 440. While FIG. 4 illustrates 5 mm as an example target protrusion 412 and a range of 1.0 mm to 10 mm as an acceptable range, it is noted that a target protrusion can be any different value, including those outside the range, based on various factors (e.g., target location to be reached, procedure to be performed, tools deployed or to be deployed, location within a subject, articulation to be performed, make/model of the endoscope, etc.). For example, the robotic system may be configured to articulate the scope 30 and sheath 40 pair such that the scope distal end 430 is maintained at about −3 mm, 0 mm, 2 mm, 2.5 mm, 3 mm, 3.5 mm, 4 mm, 5 mm (shown), 10 mm, 15 mm, or the like.

For an endoscope operating a pair of the scope 30 and the sheath 40, achieving a target protrusion can be challenging. There are many tolerances of mechanical components affecting the accuracy of position measurements of the distal ends 430, 440 including, for example, tolerances of the robotic system base 410, control assemblies 402, 404, robotic manipulators 406, 408, length of the scope 30, length of the sheath 40, etc. The tolerance stack-up can be severe. Additionally, the scope 30 and the sheath 40 have length dimensions over a meter when protrusions are measured in millimeters, a thousandths of the length dimensions. In view of the tolerances and the target granularity, attempts to provide a target protrusion at a distal end of the robotic system through control of the mechanical components at a proximal end of the robotic system is likely to miss the target protrusion and, instead, provide a negative protrusion 414 or an over-protrusion 416. The negative protrusion 414 is undesirable as it is likely to provide occluded vision and the over-protrusion 416 is undesirable as it is likely to result in sub-optimal pair driving experience. A protrusion calibration may help address these challenges.

### Protrusion Control Framework

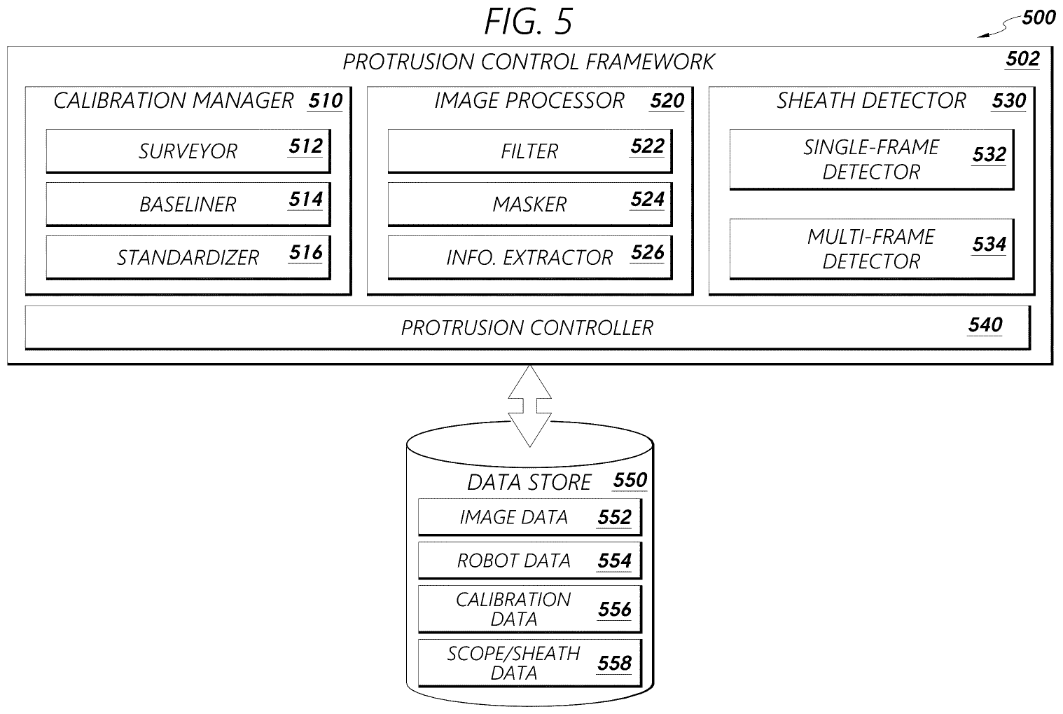

FIG. 5 illustrates an example system 500 including a protrusion control framework 502, in accordance with some implementations. The protrusion control framework 502 can be configured to calibrate a pair of a scope (e.g., the scope 30 of FIG. 2) and a sheath (e.g., the sheath 40 of FIG. 2) to a baseline protrusion from which other protrusions may be referenced. That is, regardless of tolerances of the mechanical components, the system 500 can determine a configuration that provides the baseline protrusion. Additionally, the protrusion control framework 502 can reference the baseline protrusion and control the pair to provide a target protrusion. The system 500 may be or a part of the medical system 100 of FIG. 1.

As shown, the protrusion control framework 502 can include a calibration manager module 510, an image processor module 520, a sheath detector module 530, and a protrusion controller module 540. Each of the modules can implement functionalities in connection with certain aspects of the implementation details in following figures. It should be noted that the components (e.g., modules) shown in this figure and all figures herein are exemplary only, and other implementations may include additional, fewer, integrated or different components. Some components may not be shown so as not to obscure relevant details. Furthermore, it will be understood that the architecture of the protrusion control framework 502 is modular in design and performance may be improved by improving individual modules. For example, one can improve the calibration manager module 510, the image processor module 520, the sheath detector module 530, or any component modules thereof for improved performance.

In some embodiments, the various modules and/or applications described herein can be implemented, in part or in whole, as software, hardware, or any combination thereof. In general, a module and/or an application, as discussed herein, can be associated with software, hardware, or any combination thereof. In some implementations, one or more functions, tasks, and/or operations of modules and/or applications can be carried out or performed by software routines, software processes, hardware, and/or any combination thereof. In some cases, the various modules and/or applications described herein can be implemented, in part or in whole, as software running on one or more computing devices or systems, such as on a user or client computing device, on a network server or cloud servers (e.g., Software-as-a-Service (SaaS)), or a control circuitry (e.g., the control circuitry 211, 251 of FIG. 2). It should be understood that there can be many variations or other possibilities.

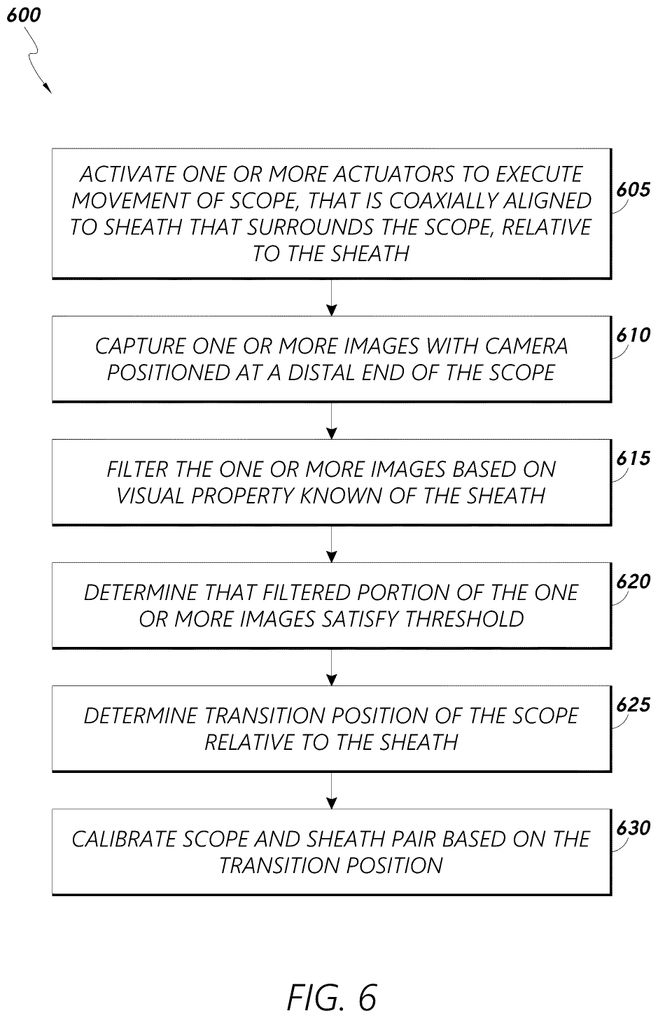

The calibration manager module 510 can be configured to execute a calibration workflow that, when successfully executed, can determine a baseline protrusion. FIG. 6 provides an example of the calibration workflow. The calibration manager module 510 can include any of an surveyor 512, a baseliner 514, and/or a standardizer 516 in connection with executing the calibration workflow.

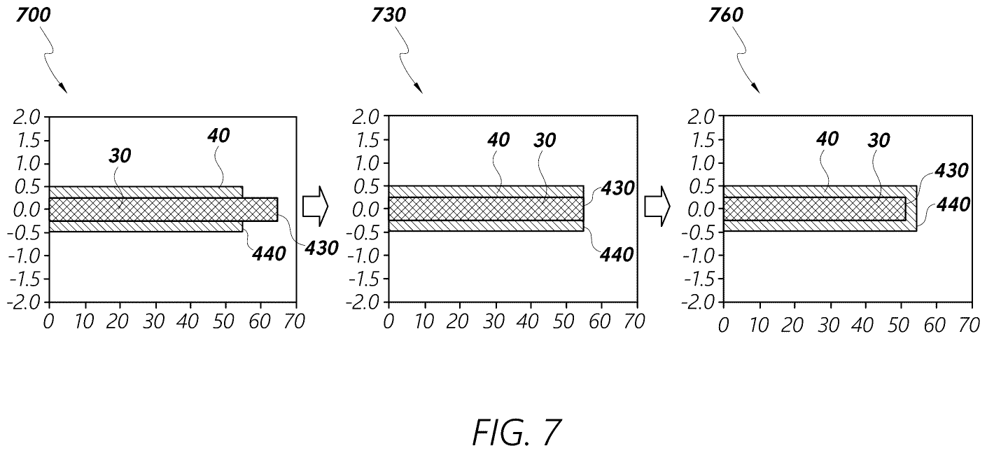

The surveyor 512 can be configured to enable sampling of different protrusions in search of a baseline protrusion by controlling position of a scope distal end (e.g., the scope distal end 430 of FIG. 4) relative to a sheath distal end (e.g., the sheath distal end 440 of FIG. 4). The protrusion controller module 540 may assist the surveyor 512 in varying protrusion through control of the scope 30 or the sheath 40. In some examples, the surveyor 512 may vary the protrusion monotonously (in the same extension/retraction direction) at a constant rate. The rate can be set based on a sampling frequency of sensor data (e.g., captured image data) used by the image processor module 520. For example, the rate can be set higher when the sampling frequency is higher and, conversely, the rate can be set lower when the sampling frequency is lower. FIG. 7 provides a detailed example of sampling protrusions for the baseline protrusion.

The baseliner 514 can be configured to examine a protrusion sampled by the surveyor 512 and, when the sampled protrusion is a baseline protrusion, configuring the system 500 to log robot data and/or kinematic data of the baseline protrusion. The baseline protrusion can be identified based on an occurrence of sample data (e.g., image data, acoustic data, EM data, etc.) satisfying one or more criteria (e.g., threshold conditions) that signal the baseline protrusion. For example, the baseline protrusion can be specified as a protrusion where a sheath opening is first detected in an image captured by a camera device proximate the scope distal end 430. In the example, images captured at sampled protrusions can be examined for the sheath detection and, when a particular image satisfies the criterion of first sheath detection, the protrusion at the time the image is captured can be identified as the baseline protrusion.

If a baseline protrusion is found, the baseliner 514 logs robot data and/or kinematic data of the sheath and the scope so that the system 500 memorizes command data or state data causing the baseline protrusion specific to the pair. Once baselined, the system 500 can provide the baseline protrusion without having to execute the calibration workflow. The baseliner 514 may implement its functionalities in connection with the image processor module 520 and the sheath detector module 530.

The standardizer 516 can be configured to optionally adjust protrusion to provide a “standard protrusion” as a part of the calibration workflow. The standard protrusion may be standard in the sense that other protrusions are measured against the standard protrusion. The standard protrusion is to be differentiated from the baseline protrusion. For example, a baseline protrusion identified by the baseliner 514 may be a non-zero protrusion (e.g., −1.2 mm, +0.35 mm, etc.). Generally, it is more desirable to control protrusions from zero protrusion (i.e., 0.0 mm) so that the system 500 may refer to any protrusion from the zero protrusion instead of the baseline protrusion. Here, the zero protrusion may be the standard protrusion from which all other protrusions are measured. Additionally, the standard protrusion is to be differentiated from a target protrusion, which can be any protrusion in a range of possible protrusions providable by the system 500. It is noted that the system 500 may accurately provide a target protrusion after either the baseline protrusion or the standard protrusion is determined.

The standardizer 516 may determine an amount to extend/retract either or both of the scope 30 and sheath 40 to accomplish the standard protrusion based on a kinematic model of the scope 30 and/or the sheath 40. For example, the standardizer 516 can compute an amount to adjust robotic manipulators (e.g., the robotic manipulators 406, 408 of FIG. 4) that would, based on the kinematic model, offset the baseline protrusion to the standard protrusion.

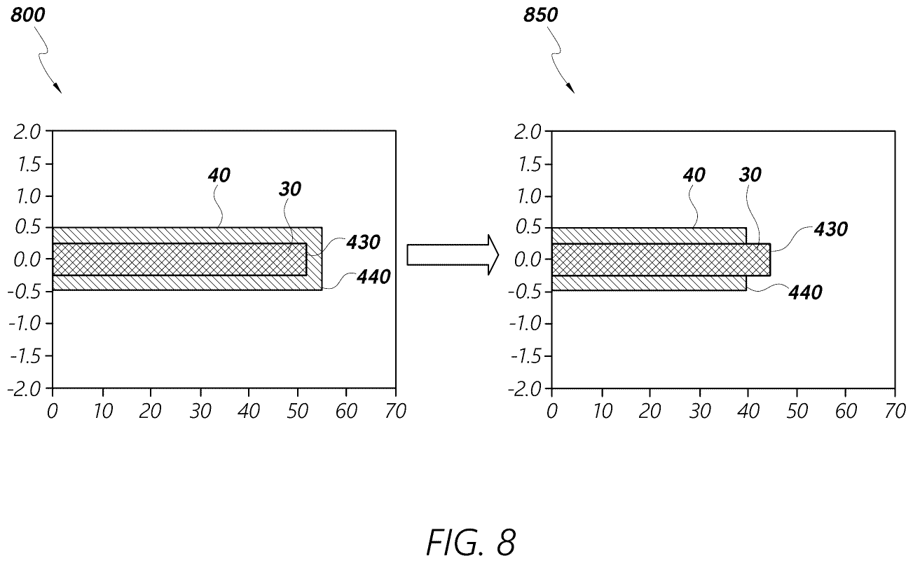

While a zero protrusion is provided as an example standard protrusion, it is noted that a standard protrusion can be any other convenient protrusion that the system 500 may want to provide at the end of the calibration workflow and use as a reference. For example, the standard protrusion can be −3 mm, 0 mm, 2 mm, 2.5 mm, 3 mm, 3.5 mm, 4 mm, 5 mm (shown in FIGS. 4 and 9B), 10 mm, 15 mm, or any other protrusion. FIG. 8 provides a detailed example of calibrating to a standard protrusion.

The image processor module 520 can be configured to access one or more images captured by an imaging device proximate the scope distal end 430 and generate various representations and/or information used by the sheath detector module 530 to detect at least one image that signals the baseline protrusion described in relation to the baseliner 514. The image processor module 520 can include a filter 522, masker 524, and information extractor 526.

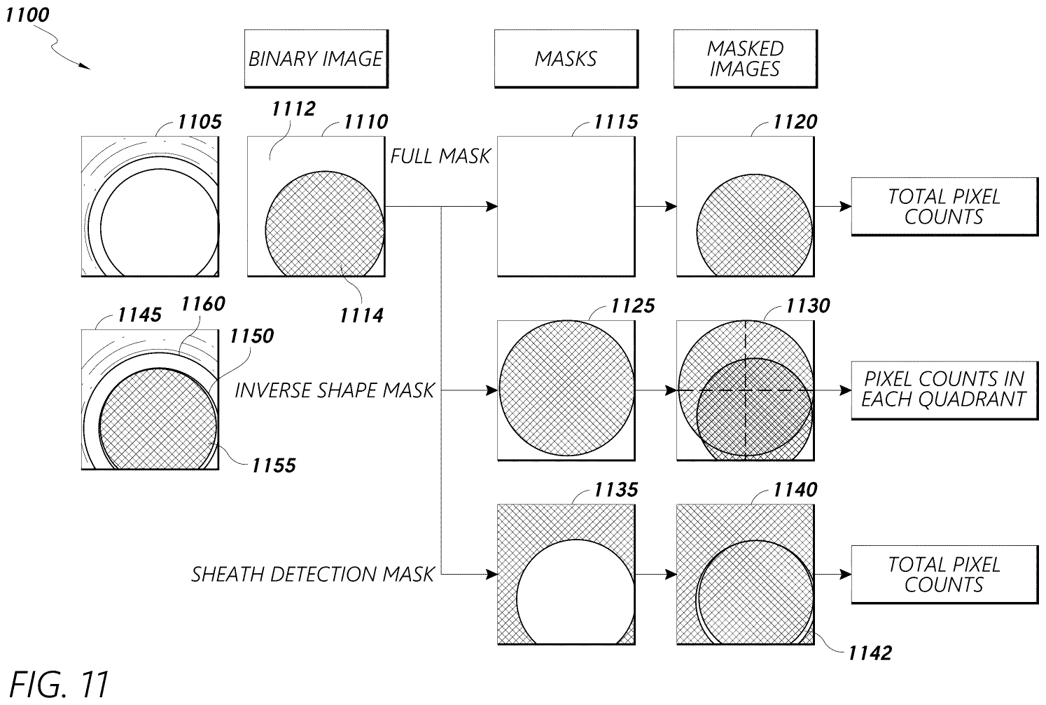

The filter 522 can be configured to remove portions of the images that are unlikely to depict/contain the sheath 40. The masker 524 can be configured to focus on a specific region of the images and further perform noise reduction in the images. The information extractor 526 can be configured to extract information, such as pixel counts, that can be compared against some criteria by the sheath detector module 530. FIG. 11 provides detailed examples of the filter 522, masker 524, and information extractor 526.

The sheath detector module 530 can be configured to perform sheath detection in the images. In some implementations, the sheath detection can involve evaluating various information extracted by the information extractor 526 against various criteria. The sheath detector module 530 can include a single-frame detector 532 and/or a multi-frame detector 534.

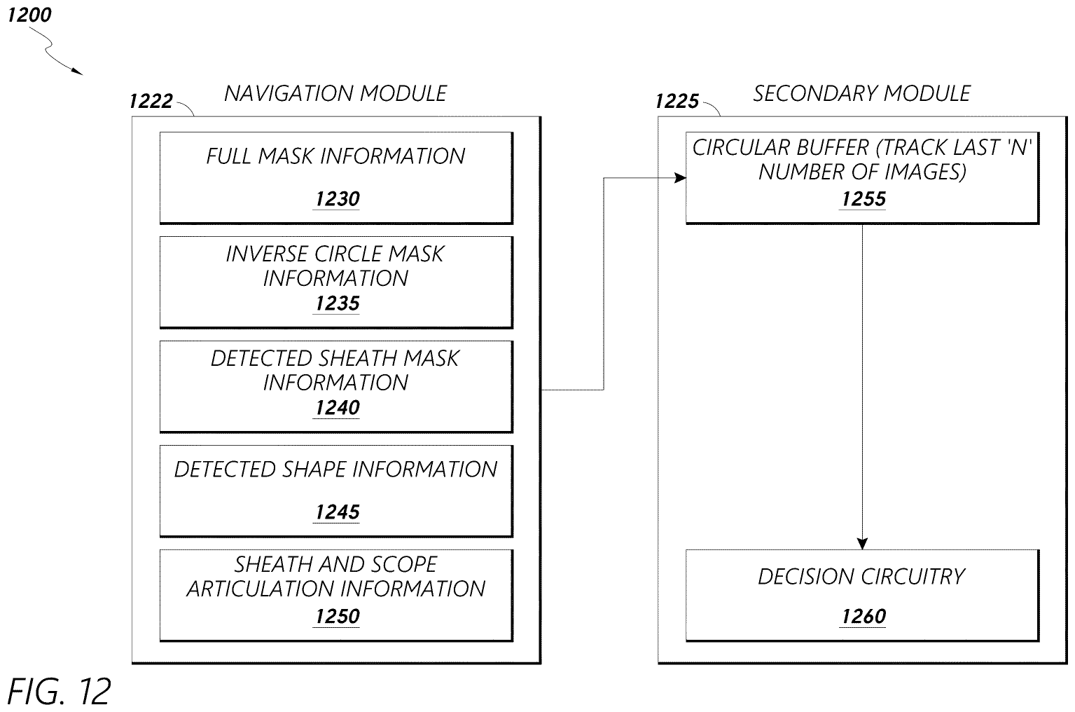

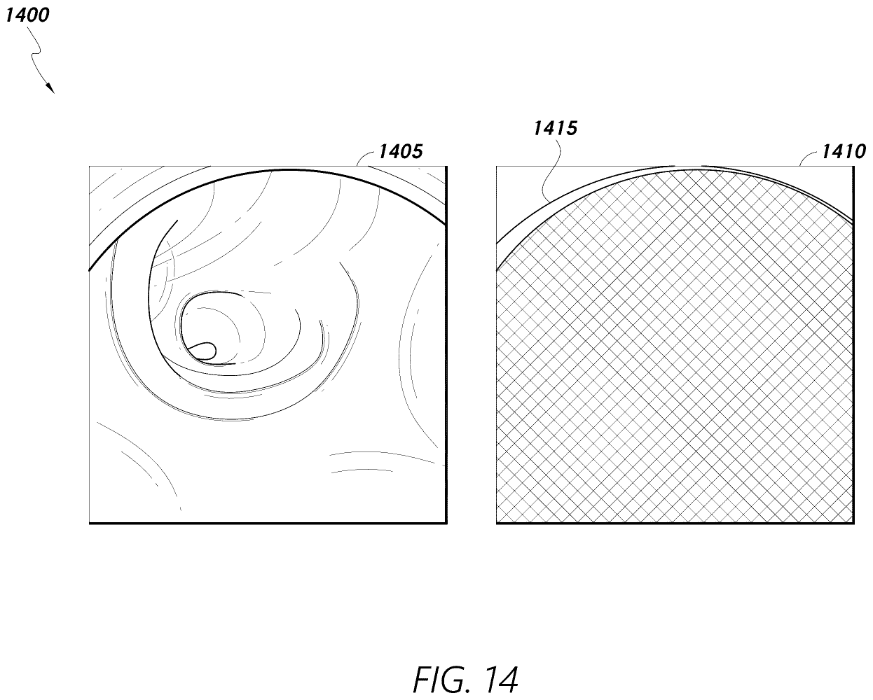

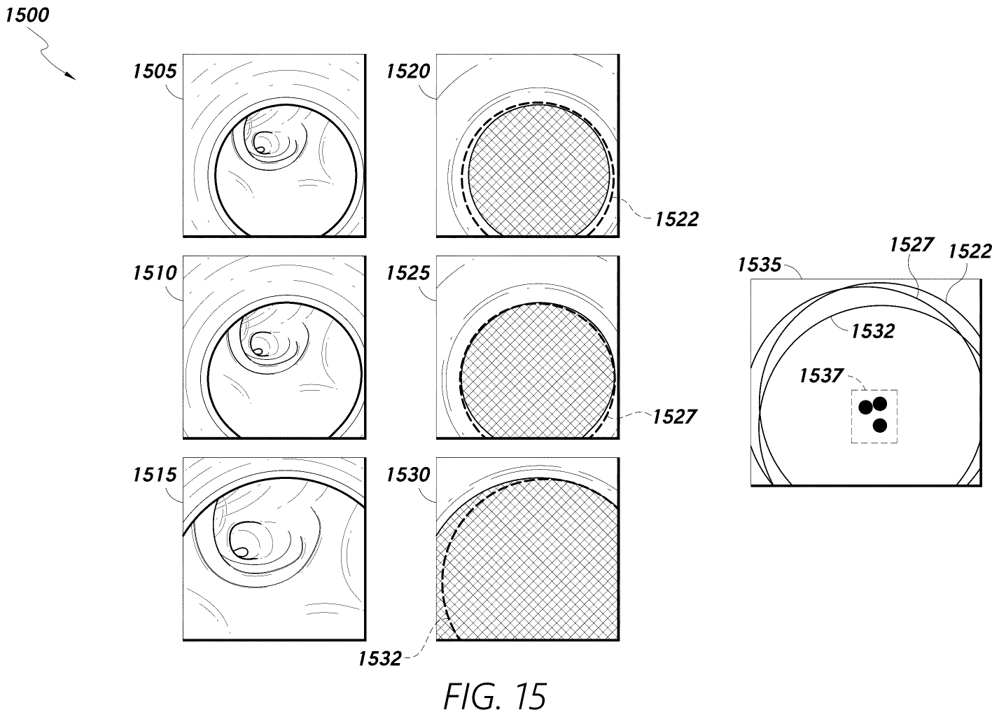

The single-frame detector 532 can be configured to detect the sheath 40 from a single captured image, which may be the latest captured image. The multi-frame detector 534 can be configured to detect the sheath 40 from multiple captured images, which may be the latest N-number of captured images. Each detector 532, 534 may have its own set of criteria (e.g., threshold conditions) for sheath detection. As will be described in greater detail in relation to FIG. 12, the single-frame detector 532 and/or the multi-frame detector 534 may not directly examine the images but, rather, compare the various information extracted by the information extractor 526 against threshold conditions to determine whether the images corresponding to the information sufficiently depicted the sheath. FIGS. 14 and 15, respectively, provide detailed examples of the single-frame detector 532 and the multi-frame detector 534.

The protrusion controller module 540 can be configured to coordinate control of the scope 30 and the sheath 40 to provide protrusion commanded by the system 500. Providing the protrusion can involve controlling the position of the scope distal end 430 relative to the sheath distal end 440 through independent or simultaneous control of the robotic manipulators 406, 408. For instance, decreasing protrusion may be accomplished by retracting the scope 30 while the sheath 40 remains stationary, extending the sheath 40 while the scope 30 remains stationary, retracting the scope 30 at a faster rate than the sheath 40 is retracted, extending the scope 30 at a slower rate than the sheath 40 is extended, or retracting the scope 30 while the sheath 40 is extended. Conversely, increasing protrusion may be accomplished by extending the scope 30 while the sheath 40 remains stationary, retracting the sheath 40 while the scope 30 remains stationary, protruding the scope 30 at a faster rate than a protruding sheath 40, retracting the sheath 40 at a faster rate than a retracting scope 30, or protruding the scope 30 while retracting the sheath 40. Constant protrusion may be accomplished by retracting or extending the scope 30 simultaneously with the sheath 40 at the same rate.

Before calibration, the protrusion controller module 540 can vary the amount of protrusion without knowing a target protrusion. In an example, the surveyor 512 can request the protrusion controller module 540 provide a monotonically decreasing protrusion. After calibration, the protrusion controller module 540 can provide the target protrusion with accuracy and precision. For example, the protrusion controller module 540 can receive a command to provide 2.3 mm protrusion and control one or both of the robotic manipulators 406, 408 to provide the commanded protrusion. Additionally, when calibrated, the protrusion controller module 540 may become able to determine a current protrusion by examining robot data of the scope 30 and the sheath 40 pair.

As shown with the example system 500, the protrusion control framework 502 can be configured to communicate with one or more data stores 550. The data store 550 can be configured to store, maintain, and provide access to various types of data to support the functionality of the protrusion control framework 502. For example, the data store 550 may store, maintain, and provide access to image data 552 including images captured for analysis by the image processor module 520. As another example, the data store 550 may store, maintain, and provide access to robot data 554 and calibration data 556. The robot data 554 can include robot command data (e.g., logs of articulation commands, protrusion commands, etc.), robot state data (e.g., logs of articulation state, protrusion state, diagnostic results, etc.), and/or robot configuration data (e.g., component identifiers, scope/sheath model, minimum adjustable protrusion, firmware version, etc.). The calibration data 556 can include a baseline protrusion, a standard protrusion, and/or robot data 554 relating to the scope 30 and the sheath 40 that accomplishes the baseline protrusion or the standard protrusion. As another example, the data store 550 may store, maintain, and provide access to scope/sheath data 558. The scope/sheath data 558 can include various properties specific to the scope 30 and the sheath 40. In some examples, the filter 522 may use some visual properties relating to the sheath 40 (e.g., such as an opening size/shape, color of an inner wall of the opening, or the like) to remove portions of the image data 552 that are unlikely to depict/contain the sheath 40. In some examples, the standardizer 516 may use some of functional or structural properties relating to the scope 30 (e.g., lighting capabilities, a field-of-view or image resolution of an imaging device of the scope 30) or the pair (e.g., known dimensions of the scope 30 and sheath 40) to determine the calibration data 556 that, when commanded, would adjust the baseline protrusion to the standard protrusion. It is noted that the data store 550 may be any of the computer-readable media described above, which include volatile/temporary memories.

### Protrusion Calibration Workflow

FIG. 6 is a flow diagram of an example process 600 for calibrating protrusion of a scope (e.g., the scope 30 of FIG. 2) and sheath (e.g., the sheath 40 of FIG. 2) combination, in accordance with some implementations. The process 600 may be executed to determine a position of a scope distal end (e.g., the scope distal end 430 of FIG. 4) relative to a sheath distal end (e.g., the sheath distal end 440 of FIG. 4). In various examples, the process 600 is implemented to calibrate an endoscopic device prior to insertion of an endoscope into a subject or as needed intraoperatively. The calibration manager module 510 of FIG. 5 may execute the process 600.

At block 605, the process 600 may activate one or more actuators (e.g., move the manipulators 406, 408 of FIG. 4) to execute a movement of the scope relative to the sheath. The movement may be an extension or retraction of the scope 30 relative to the sheath 40.

The movement in this block 605 is in a direction that changes the direction of the protrusion. For example, if the scope 30 and sheath 40 pair was in an original state of positive protrusion, the movement is directed toward providing negative protrusion. Conversely, if the pair was in an original state of negative protrusion, the movement is directed toward providing positive protrusion.

In some examples, it may be advantageous to change protrusion from positive to negative by retracting the scope 30 while the sheath 40 remains stationary (as opposed to extending the sheath 40 while the scope remains stationary). The retraction can help avoid unintentional damage to the scope 30 during the movement and, where the process 600 is carried out intraoperatively to re-calibrate, the retraction may better utilize limited space within a subject. Accordingly, in the interest of brevity, the process 600 is described with focus on retracting the scope distal end 430 into the sheath 40 but one skilled would appreciate that the process 600 can be altered to execute extension of the scope 30 such that the scope distal end 430 exits the sheath distal end 440.

At block 610, the process 600 may, during the movement of the scope 30 in relation to the sheath 40 (e.g., the movement executed at block 605), capture one or more images with a camera (e.g., the cameras 48 of FIG. 2). In various examples, images may be taken continuously at regular intervals or irregular intervals as the scope distal end 430 is retracted. At some point, the retraction will cause an opening of the sheath distal end 440 to be made visible in the one or more images. In some examples, the surveyor 512 of FIG. 5 may execute the movement and capture the images.

At block 615, the process 600 may filter the one or more images based on one or more visual properties known of the sheath 40. The visual properties may include color (hue, saturation, and/or value defining a color), contrast, shape, size, thickness, pattern, marker, notch, curvature, or the like. In some examples, the filtering may involve extracting or otherwise identifying a portion of the one or more images that correspond to the sheath distal end 440. For example, if inside of the sheath distal end 440 has a known pattern, marking, or color, the filtering can extract or otherwise identify that portion. As another example, if inside of the distal end is expected to show certain curvature (as a separate visual property or as a visual property combined with another property, such as thickness), the filtering can extract or otherwise identify a portion showing the curvature.

In some cases, prior to the process 600, internal walls of the distal end of the sheath may be configured to emphasize one or more visual properties used in the filtering to facilitate such filtering. For instance, where the endoscope is expected to traverse a bronchial lumen that is mostly red/pink in color under a lighting (e.g., by the one or more lights 49 of FIG. 2), the internal wall may be colored with blue or the complementary color (green) so that it is distinguishable from other parts of the image. In other examples, the internal walls may be coated with phosphors such that the distal end of the sheath may be identified with its luminous effect. Many variations are possible. In some examples, the filter 522 in FIG. 5 may filter the images.

At block 620, the process 600 may determine that a filtered portion of the one or more images satisfies a threshold condition. For instance, the threshold may be that the number of pixels of the filtered portion of the one or more images be greater than a percentage of the total number of pixels. In an example, the threshold may be set based on a visible shape of an opening of the sheath distal end 440. For instance, the threshold may be set as the circumference of the opening of the sheath distal end 440 multiplied by a scalar value and compared to the total number of filtered pixels of the image. As another example, the threshold condition may be whether a radius of a curvature detected in the filtered portion is less than a threshold radius. As yet another example, the threshold condition may be whether a number of concentric rings, notches, or other patterns/markers detected in the internal walls is above a threshold number. Various threshold mechanisms may be used to determine whether the threshold condition is met. In some examples, the sheath detector module 530 of FIG. 5 may determine whether the threshold condition is satisfied or not.

When the threshold condition is satisfied, the sheath 40 may be deemed detected. Conversely, while the threshold condition is not satisfied, the sheath 40 may be deemed not detected. When the sheath 40 is detected, the process 600 can stop movement of both the scope 30 and the sheath 40. When the sheath 40 is not detected, the process 600 may continue retracting the scope distal end 430 (or advancing the sheath distal end 440) until some other stopping condition (e.g., a detection failsafe condition) is satisfied.

At block 625, the process 600 may determine a transition position of the scope 30 relative to the sheath 40 based on the determining in block 620. The transition position may refer to a relative position between the scope distal end 430 and sheath distal end 440 where the sheath distal end 440 transitions between being detected and not being detected, or vice versa. The transition position can be the baseline protrusion described in relation to the baseliner 514 in FIG. 5.

At block 630, the process 600 may calibrate the scope 30 and the sheath 40 pair based on the transition position. Since the scope distal end 430 must be retracted to within the sheath 40 for the sheath distal end 440 to be visible to the camera proximate the scope distal end 430, the process 600 may ascertain that the scope distal end 430 is recessed in the sheath 40 at the transition position by an offset. The offset may be determined or supplied based on prior measurements or known properties (e.g., the scope/sheath data 558 of FIG. 5) of the scope 30 and sheath 40. For instance, the process 600 may determine that the scope 30 should negatively protrude the sheath 40 by 2.1 mm offset at the transition position based on manufacturing models or known dimensions of the scope 30 and sheath 40.

Based on the transition position and the determined offset, a target protrusion may be provided. It is noted that movement of both the scope 30 and sheath 40 was stopped at the end of block 620. If the target protrusion is zero protrusion, the scope distal end 430 may be extended (or the sheath distal end 440 be retracted) by the offset at the transition position to provide zero protrusion. That is, in the 2.1 mm offset example above, the scope 30 can be extended by 2.1 mm to provide zero protrusion. If the target protrusion is 3.5 mm positive protrusion, the scope distal end 430 can be extended by additional 3.5 mm from the zero protrusion. If the target protrusion is 10 mm negative protrusion, the scope 30 can be retracted by 7.9 mm from the transition position. The standardizer 516 of FIG. 5 may determine the offset and provide the target protrusion as its standard protrusion. FIG. 8 provides example calibration performed at block 630.

While the process 600 focuses on images captured with a camera, it is contemplated that other types of sensors capable of detecting retraction of the scope distal end 430 into the sheath 40 or exit of the scope distal end 430 from the sheath 40 may be used. For example, an acoustic sensor (e.g., an ultrasound sensor) proximate the scope distal end 430 or the sheath distal end 440 may detect change in reverberations when the scope distal end 430 passes by the sheath distal end 440.

### Retraction and Extension During Calibration

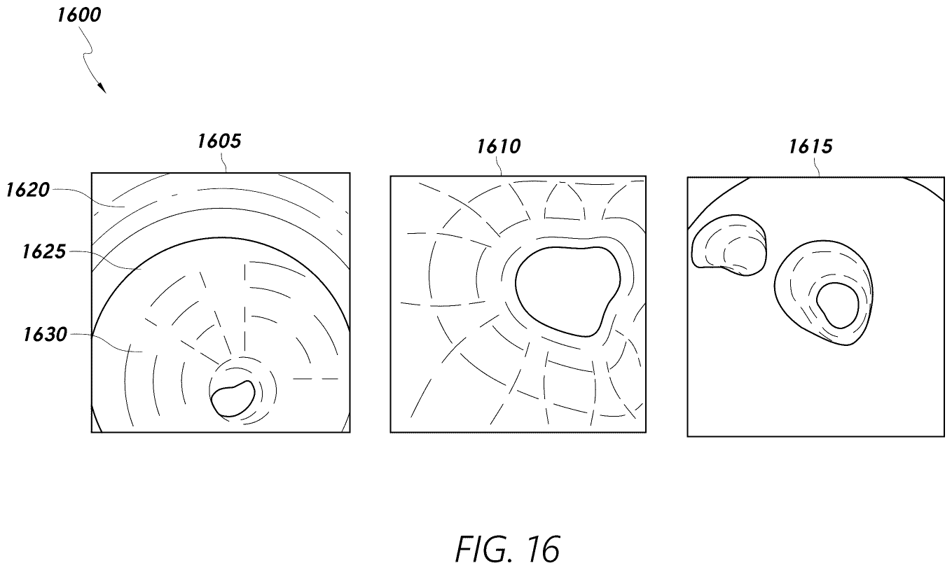

FIG. 7 is a set of illustrations 700 of cross sections of a scope (e.g., the scope 30 of FIG. 2) and sheath (e.g., the sheath 40 of FIG. 2) pair at a distal portion of an endoscope during transition position determination, in accordance with some implementations. Before calibration, a scope distal end (e.g., the scope distal end 430 of FIG. 4) may be positioned at an unknown position relative to a sheath distal end (e.g., the sheath distal end 440 of FIG. 4). In some examples, the scope 30 may be extended to make certain that the scope distal end 430 is out of the sheath 40. The distance to extend here may be determined based on system tolerances and/or known over-protrusion limit. For example, 36 mm extension of the scope 30 would be sufficient extension for a pair with known tolerance range of −4 mm to 32 mm. In some other examples, the extension can be performed until a sheath is confirmed to be not visible, such as a transition from a detected sheath image 1605 to no detection image 1610 in FIG. 16.

A first illustration 700 shows the scope 30 and sheath 40 prior to calibration. In this example, the scope distal end 430 is protruding from the sheath distal end 440. The sheath distal end 440 is not visible to a camera situated proximate the scope distal end 430 and the camera cannot capture any portion of the sheath 40. Calibration may involve retracting the scope until the sheath 40 is made detectable in one or more images captured by a camera proximate the scope distal end 430.

In various examples, the speed of the movement between the scope and the sheath may be varied. For example, in various examples, the scope 30 may be moved twice as fast as the sheath 40. The varying rates of movement between the scope 30 and the sheath 40 may allow the system to achieve a calibrated position in a shorter period of time than keeping the rates of movement constant. The respective rates may take into consideration of safety of the subject, integrity of the sheath 40 and the scope 30, and phase of a procedure. As one example, it would be more appropriate to use faster speed while the scope 30 is within the sheath 40 since the scope 30 is protected by the sheath 40. In some examples, the respective rates may be determined based on image capturing frequency of a camera so that the transition position is not missed by a disproportionately fast rate compared to the frequency. The reduction in the time to a calibrated position will save time for the physician and the patient.

In a second illustration 730, the scope 30 is retracted from the position in the first illustration 700 to a position where the scope distal end 430 and the sheath distal end 440 are aligned (zero protrusion). Still, the camera situated proximate the scope distal end 430 is unlikely to capture an image depicting a portion of the sheath 40 at the zero protrusion. Thus, retraction of the scope 30 continues. In some examples, the position at which the scope 30 and sheath 40 are aligned may be referred to as an exit position.

A third illustration 760 shows a negative protrusion where the sheath distal end 440 is protruding beyond the scope distal end 430. Here, a camera proximate the scope distal end 430 may be capable of capturing an image depicting an inner lumen of the sheath distal end 440. Based on the image, the baseliner 514 of FIG. 5 can identify a transition position and calibrate the scope 30 and the sheath 40.

FIG. 8 is a set of illustrations of cross-sections of a scope (e.g., the scope 30 of FIG. 2) and sheath (e.g., the sheath 40 of FIG. 2) pair at a distal portion of an endoscope during calibration, in accordance with some implementations. A first illustration 800 shows the scope 30 retracted to a negative protrusion where a scope distal end (e.g., the scope distal end 430) is covered by the sheath 40. The negative protrusion may be just after determination of a transition position at block 625 of FIG. 6 and correspond to the third illustration 760 of FIG. 7.

The scope distal end 430 may be calibrated to a standard protrusion. A second illustration 850 shows the scope 30 moved relative to the sheath 40 such that the scope distal end 430 is protruding beyond a sheath distal end (e.g., the sheath distal end 440) to the standard protrusion. The standard protrusion may be provided at block 630 of FIG. 6 by the standardizer 516 of FIG. 5.

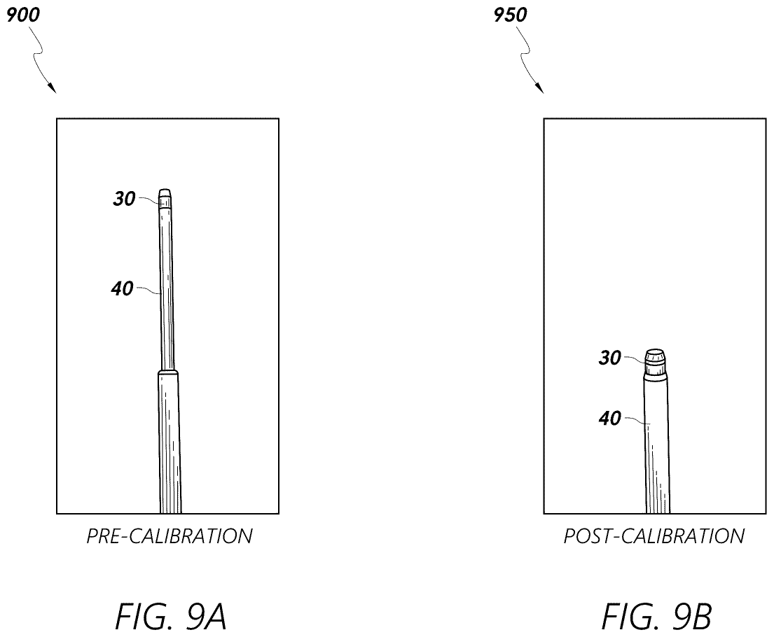

FIGS. 9A-9B are illustrations of a scope (e.g., the scope 30 of FIG. 2) and a sheath (e.g., the sheath 40) pair at pre-calibration and post-calibration, in accordance with some implementations. FIG. 9A is an illustration 900 of the pair in a pre-calibration state, where a position of the scope distal end (e.g., the scope distal end 430 of FIG. 4) may be calibrated relative to a sheath distal end (e.g., the sheath distal end 440 of FIG. 4).

FIG. 9A shows the scope 30 positioned relative to the sheath 40 such that the scope distal end 430 is protruding at, for example, approximately about 35 mm beyond the sheath distal end 440. The pair shown in FIG. 9A may be calibrated according to the process 600 of FIG. 6 disclosed herein.

FIG. 9B is an illustration 950 of the sheath 40 and the scope 30 pair in a post-calibration state, where the scope distal end 430 is at a standard protrusion. In some instances, the standard protrusion may be referred as a default protrusion. In some examples, commanded protrusions (e.g., target protrusions) may be measured from the standard protrusion. For example, assuming the pair is calibrated to have a 5 mm standard protrusion, if the original 9 mm protrusion is to be provided, a +4 mm protrusion may be commanded to reflect the assumed 5 mm standard protrusion as a reference. It is noted that the standard protrusion of 5 mm is exemplary and any other lengths of standard protrusion are possible. Once calibrated, any protrusion may be reverted to the standard protrusion without having to re-execute the process 600.

### Sheath Detection

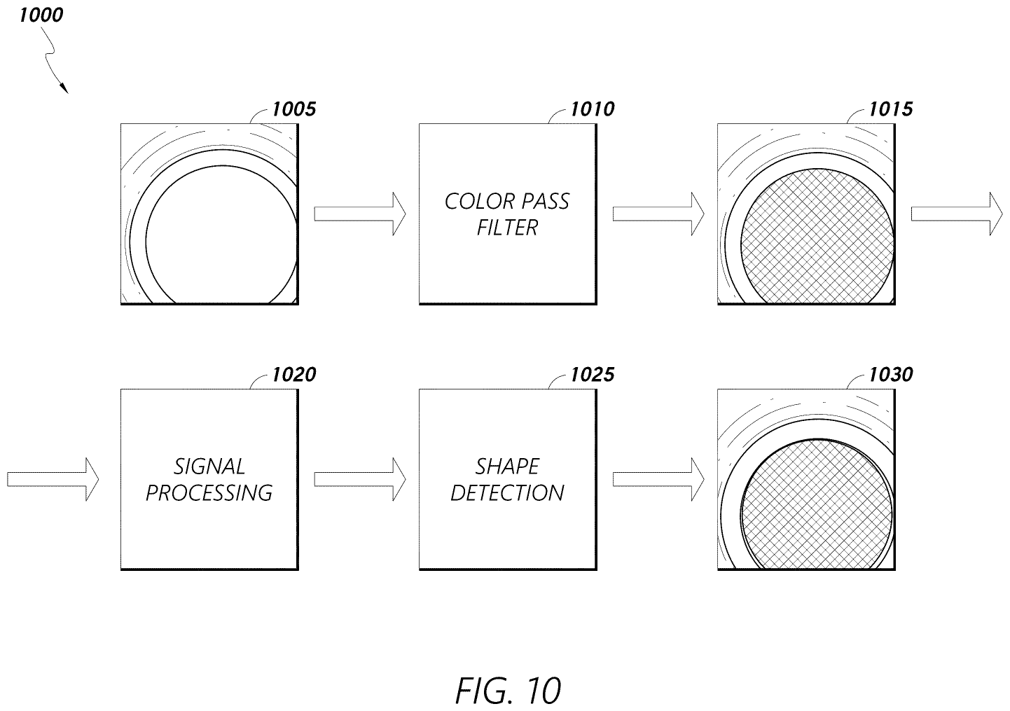

FIG. 10 illustrates an example process 1000 for detecting an inner lumen of a sheath (e.g., the sheath 40 of FIG. 2) from an image, in accordance with some implementations. The detection of the inner lumen or an opening of the sheath 40 will be referred as “sheath detection.” The sheath detector module 530 in connection with the image processor module 520 in FIG. 5 may implement the sheath detection.

At block 1005, the camera captures an image or a captured image is otherwise provided/accessed. In some examples, a lighting (e.g., by the one or more lights 49 of FIG. 2) may be tuned to reduce blurriness or overexposure possible in the captured image.

At block 1010, the image is processed with a color pass filter (e.g., a blue pass filter) configured to pass colors that are the same or substantially similar with the inner lumen color. The color pass filter may filter based on any of any of hue, saturation, or value defining a color to isolate a portion of the sheath corresponding to the inner lumen. Here, it is assumed here that the color of the inner lumen of the sheath is blue. However, other colors, such as but not limited to, brown, green, yellow, red, purple may be used as the color of the inner lumen. In some examples, the color of the inner lumen may be shown to a camera (e.g., the camera 48 of FIG. 2) prior to the process 1000 so that the camera can determine the color based on stored numerical values representing the color. In other examples, a user may input the color of the sheath 40.

At block 1015, a filtered image is generated based on the color pass filter. As shown, the filtered image eliminated (as indicated with the checkered pattern) portions of the image that were not the color of the sheath. The elimination (or filtering) can involve assigning a default color to the filtered-out portion. For example, the filtered-out portion may be assigned entirely black, white, or another color to indicate null content.

At block 1020, the filtered image may be processed to determine whether a threshold condition is satisfied, where satisfaction of the threshold condition indicates presence of a portion depicting the sheath 40. As an example threshold condition, a total number of remaining pixels after the filtering allegedly depicting the sheath 40 may be compared against a threshold pixel count. If the threshold condition is satisfied, the image may be deemed to depict the sheath 40. Various threshold conditions may be used at this block.

At block 1025, the filtered image may be further processed to detect a shape of an opening of the sheath distal end in the image. The shape is likely circular (e.g., circle, oval, ellipse, almond, egg, racetrack, etc.) but other shapes are also contemplated. Assuming a circular sheath opening, a view from within the sheath 40 would have a circular boundary in captured images.

Various image processing techniques may be implemented to detect the circle. For example, an inverse circular mask may be implemented to determine a number of pixels in a circle shape that were not filtered by the color pass filter. The threshold condition may be satisfied based on the total number of pixels within the circle and the number of pixels outside the circle. For example, a threshold condition may be set to 45%. An image with a circle that contains a number of pixels that are greater than 45% of the total number of pixels in the image will meet the threshold condition. A size and position of the circle may be determined whenever the threshold condition is satisfied based on the number and location of the pixels within the circle. For example, a threshold condition may be met when a number of pixels inside the circle exceeds a set number or percentage of pixels in the image and a center of the circle is located within a set distance from the center of the image.

A detected circle may be analyzed for parameters describing its shape. For example, a radius/diameter/curvature of the circle and a center position of the detected circle can be determined.

At block 1030, the detected shape can be analyzed against an expected shape estimated based on a known structure of the sheath 40. If the sheath 40 has a circular opening, the size (diameter or radius) of the sheath opening can be known prior to calibration and/or input into the system, thus the distance of the camera to the opening of the sheath may be determined based on a size of the detected circle in comparison to the known size sheath opening. For instance, an angle made by the center of the circle to the center of the camera to the edge of the circle may be correlated to a number of pixel lengths to make a radius of the circle. The correlation may be dependent on the specifications of the camera. The distance of the camera to the opening of the sheath may be determined from the radius of the sheath opening divided by a sine function of the angle. The distance can indicate protrusion.

### Image Data Information Extraction

FIG. 11 is an example diagram 1100 showing various filters and/or masks applied to images to extract various information used for sheath detection, in accordance with some implementations. The image processor module 520 of FIG. 5 may implement the filtering, masking, and extraction in the diagram 1100.

An unfiltered image 1105 and a filtered image 1145 are provided as references. The unfiltered image 1105 shows an outline of a sheath opening indicating that a distal end of a scope is retracted within a sheath. The filtered image 1145 shows a true sheath perimeter 1150, which may not exactly coincide with a perimeter of the detected shape 1155, and remaining artifacts 1160 such as different gradations/hues of the passed color.

The binary image column shows generation of a binary image 1110 that removes the artifacts 1160 to provide monotonous filtered-in portion 1112 and a filtered-out portion 1114, where the artifacts 1160 are eliminated. The binary image 1110 can facilitate masking operation and pixel counting operation that will be provided in the following masks column and masked images column.

The masks column illustrates a set of masks: a full mask 1115, an inverse shape mask (e.g., an inverse circular mask) 1125, and a sheath detection mask 1135. These masks may be used to help determine whether one or more sheath detection threshold conditions are satisfied. The shown masks are exemplary and other masks having other shapes may be used as appropriate depending on the known shape of the opening of the sheath as seen from within. In FIG. 11, blank areas are to be passed through a mask and patterned areas are to be blocked by the mask. After the masks 1115, 1125, 1135 are applied to the binary image 1110, corresponding images 1120, 1130, 1140 are generated. Pixels can be counted on the corresponding images 1120, 1130, 1140 for threshold condition comparison described in relation to FIGS. 5, 14, and 15.

The full mask 1115 includes the entire area of the image and allows pass through of every pixel. When the full mask 1115 is applied to the binary image 1110 (performing a pixelwise Boolean AND on all pixels), the resulting fully masked image 1120 is the same as the binary image 1110. For the fully masked image 1120, a total number of pixels in the blank area is counted.

The inverse circular mask 1125 masks out a circle such that pixels in the circle are removed from counting. The circle of the inverse circle mask 1125 may be positioned at a fixed position. For example, assuming the binary image 1110 has known dimensions of a square, the circle may have its center positioned at the center of the square with a diameter matching a side of the square. When the inverse circle mask 1125 is applied to the binary image 1110, the resulting inverse circle-masked image 1130 additionally removes pixels corresponding to the circle from the binary image 1110. In some instances, application of the inverse circular mask 1125 can result in a tilted 8-like image. For the inverse circle-masked image 1130, total numbers of pixels in each quadrant of the blank area are counted.

The sheath detection mask 1135 may be a mask of the sheath as detected by another mean. For instance, along with the color pass filter used in detecting the opening of the sheath, a machine learning model or other segmentation techniques may be used to provide a segmentation mask for the opening as the sheath detection mask 1135. If the machine learning model is highly accurate, the sheath detection mask 1135 may be close to the true sheath perimeter 1150 of the filtered image 1145.

When the sheath detection mask 1135 is applied to the binary image 1110, the resulting sheath-masked image 1140 can “eclipse” and leave Baily's bead-like thin outline 1142 corresponding to the remaining perimeter of the opening. For the sheath-masked image 1140, a total number of pixels in the thin outline 1142 is counted.

### Example Sheath Detection System

FIG. 12 is an example block diagram 1200 of a sheath detection system, in accordance with some implementations. In an example, the sheath detection system includes a navigation module 1222 configured to command and control articulation of an endoscope. The navigation module 1222 may have an initialization workflow that can involve, for bronchoscopy, exemplary steps of: (1) correcting roll, (2) touch main carina, (3) retract by a first predetermined distance (e.g., 40 mm), (4) insert into a bronchus, (5) retract by a second predetermined distance, and (6) complete navigation initialization. The protrusion calibration (e.g., the process 600 of FIG. 6) may be performed within the initialization workflow, for example during the (3) retraction by the first predetermined distance. Accordingly, in this example, the protrusion calibration does not add another step to the initialization workflow.

The navigation module 1222 can implement filtering and masking described in FIG. 12. As shown, the navigation module 1222 may access or provide data related to full mask information 1230, inverse circle mask information 1235, detected sheath mask information 1240, detected shape information 1245, and sheath and scope articulation information 1250.

The full mask information 1230 relates to the total number of pixels counted for the fully masked image 1120 of FIG. 11. The inverse circular mask information 1240 relates to pixel counts of each of the quadrants in the inverse circle-masked image 1130 of FIG. 11. The detected sheath mask information 1240 relates to the total number of pixels counted in the thin outline 1142 for the sheath-masked image 1140 of FIG. 11. The detected shape information 1245 relates to whether a circle was detected, the center position, and/or radius of the circle determined at block 1025 of FIG. 10. The sheath and scope articulation information 1250 relates to commanded articulation and/or current articulation state.

A secondary module 1225 may interface with the navigation module 1222 and support the navigation module 1222 in connection with sheath detection. The secondary module 1225 may receive data (e.g., various information 1230, 1235, 1240, 1245, 1250) from the navigation module 1222 for every (e.g., every 1, every 2, every ‘N’) or selected camera image and run a decision circuit 1260 based on the received data. The secondary module 1225 may maintain a buffer, such as a circular buffer 1255, that keeps track of the data from the last N number of images. For example, where N is set to “20”, the circular buffer 1255 may track/keep information of the last 20 images from the navigation module 1222. In some examples, the secondary module 1225 may compute some derived property from the tracked data from the last N number of images., such as a rate of change in pixels of each quadrants, etc. In those examples, the derived property may be used as part of threshold condition determinations.

The decision circuit 1260 can perform sheath detection using the tracked information in the buffer. In some examples, the decision circuit 1260 may execute a single-frame (N=1) sheath detection and/or a multi-frame (N>1) sheath detection. For sheath detection, different threshold conditions may be analyzed for the single-frame approach as compared to the multiple-frame approach. Each sheath detection approaches are described in detail below.

### Calibration Decision Process

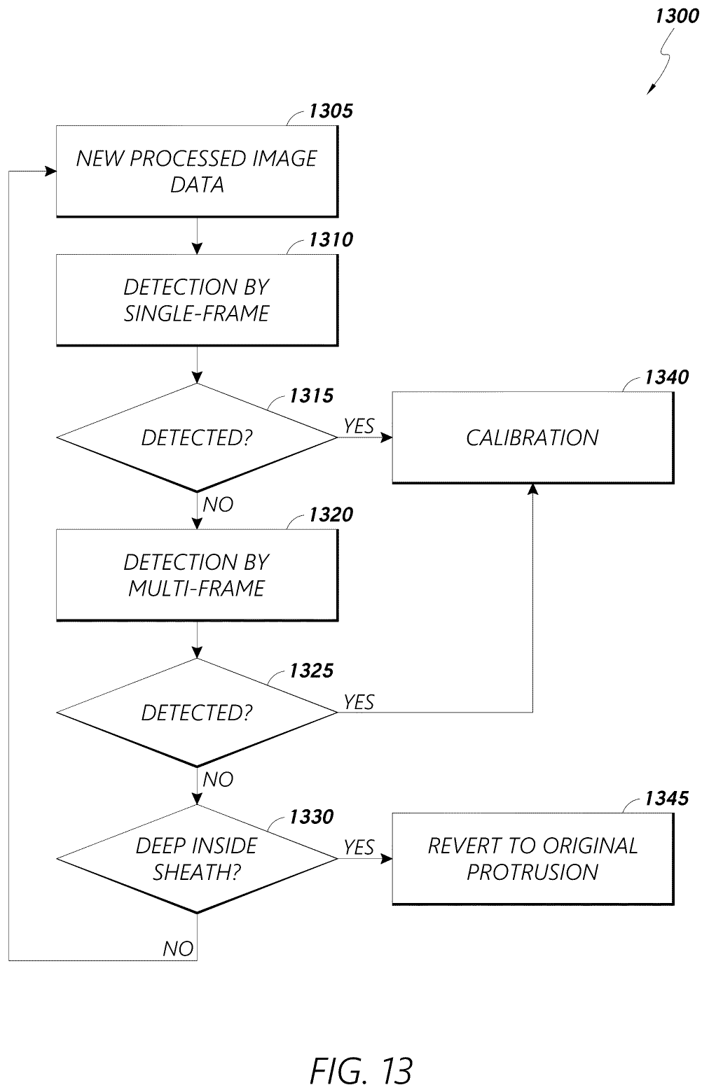

FIG. 13 is an example flow diagram of a calibration decision process 1300 involving multiple approaches, in accordance with some implementations. The calibration decision process 1300 may be implemented on or executed by the decision circuity 1260 of FIG. 12. The process 1300 may be used to determine the presence of a sheath (e.g., the sheath 40 of FIG. 2) based on image data captured by camera proximate a scope distal end (e.g., the scope distal end 430 of FIG. 4).

At block 1305, the process 1300 receives new processed image data. The processed image data may be the information 1230, 1235, 1240, 1245, 1250 described in relation to FIG. 12 from a set of N processed images. In various examples, the new processed image data may be processed data from a single image that is added to a collection of processed image data that is already stored. The single image may be the last/latest captured image.

At block 1313, the process 1300 determines whether the sheath is detected based on the processed image data from a single image. In an example, the single-frame approach and its criteria disclosed in relation to FIG. 14 are used to determine whether the sheath is detected based on the single image.

At block 1315, the process 1300 proceeds to calibration 1340 (e.g., the process 600 for calibrating protrusion in FIG. 6) if the sheath was detected by the single-frame approach at block 1310. If the sheath was not detected, the process 1300 may continue analyzing the image data at block 1320.

At block 1320, the process 1300 determines whether the sheath is detected based on processed image data from the last N images. In the example shown in FIG. 15, N is set to 3 images but N may be set to any number of images. In an exemplary example, the N last images are analyzed according to the multi-frame approach and its criteria disclosed in relation to FIG. 15 herein.

At block 1325 of the process 1300, the process 1300 proceeds to the calibration 1340 if the sheath was detected by the multi-frame approach at block 1320. If the sheath was not detected at block 1325, the process 1300 may continue to block 1330 to determine whether a “deep inside sheath” condition has been detected at block 1330. The “deep inside sheath” condition is a situation when the scope has retracted too far into the sheath without detection of the sheath.

At block 1330, the process 1300 can perform the “deep inside sheath” detection. In some examples, the “deep inside sheath” detection can be a failsafe algorithm that would stop the scope from retracting indefinitely when both the single-frame approach and the multi-frame approach fail to detect the sheath.

In some implementations, the “deep inside sheath” detection may be based on a threshold condition that would monitor how far the scope should have retracted in relation to the sheath and would stop when it has retracted beyond a retraction threshold. The retraction threshold may be a maximum retraction distance (e.g., 40 mm retraction) of the scope based on where the scope started or may be based on an expected change in negative protrusion (e.g., −30 mm change in protrusion). In some examples, the retraction threshold may be determined based on non-distance metrics. For example, the retraction threshold may be based on a total count of the number of pixels that have a specific hue/saturation which may be deemed satisfied when the total count exceeds a certain pixel count (e.g., over 90% of the pixels are identified as black).

If the threshold condition is satisfied, the process 1300 may proceed to block 1345 to abort calibration and/or revert the scope to its original protrusion. If the threshold condition is not satisfied, the process 1300 may loop back to block 1305 to wait for new processed image data.

### Single-Frame Sheath Detection Approach

FIG. 14 is a set of images 1400 showing a single-frame approach. In some examples, the single-frame detector 532 of FIG. 5 may execute the single-frame approach. On the left side is a captured image 1405 and a shape-detected image 1410 showing a detected shape (circle) 1415 of the captured image 1405. The shape-detected image 1410 may be analyzed to determine whether it satisfies a threshold condition that indicates presence of the sheath in the captured image 1405.

The captured image 1405 may depict the moment that the scope is retracting into the sheath during a calibration. The image processor module 520 of FIG. 5 may have processed the captured image 1405 to extract various information 1230, 1235, 1240, 1245, 1250 of FIG. 12 for the captured image 1405. The single-frame approach can involve comparing the information 1230, 1235, 1240, 1245, 1250 to one or more criteria. In some examples, the one or more criteria can be the following thresholds conditions.

One threshold condition may determine whether any of the pixel count in one of the inverse circular quadrants accessed from the inverse circle mask information 1235 exceeds (greater than) a first threshold value. In some implementations, the first threshold value may be, about 5% of the total number of pixels in the quadrant, about 10% of the total number of pixels in the quadrant, about 15% of the total number of pixels in the quadrant, about 20%, etc.

Another threshold condition may determine whether the total number of pixels in a thin outline accessed from the detected sheath mask information 1240 falls short of (less than) a second threshold value (this threshold value may be referred as a first limit).

Yet another threshold condition may determine whether a radius of the detected circle accessed from the detected shape information 1245 exceeds a third threshold value. The third threshold value may be a length of radius measurable in pixels. For instance, where the image is 128 pixels by 128 pixels, a radius of the circle that measures about 102 pixels would be a radius length of approximately 80% of a side of the image.

Yet another threshold condition may determine whether the total number of pixels accessed from the full mask information 1230 falls short of a fourth threshold value (a second limit).

In addition to the threshold conditions, the single-frame approach may determine whether a condition that the captured image 1405 analyzed was the first image in which a circle was detected is satisfied. If all of the conditions above are satisfied, the single-frame approach may determine that the sheath is detected.

It is noted that one or more threshold conditions may be dropped in implementations with less strict sheath detection criteria. Conversely, one or more threshold conditions may be added in implementations with more strict sheath detection criteria.

### Multi-Frame Sheath Detection Approach

FIG. 15 illustrates a set of images 1500 showing a multi-frame approach, in accordance with some implementations. In some examples, the multi-frame detector 534 of FIG. 5 may execute the multi-frame approach. The multi-frame approach may analyze information (e.g., information 1230, 1235, 1240, 1245, 1250 accessed from the circular buffer 1255 in FIG. 12) from multiple previous frames to determine if a sheath is detected.

In some implementations, the multiple-frame approach may be utilized as a fallback approach for when the single-frame approach fails to detect the sheath. The set of images 1500 illustrate 3 consecutive frame pairs: a first image 1505 paired with a corresponding first shape-detected image 1520, a second image 1510 paired with a corresponding second shape-detected image 1525, and a third image 1515 paired with a corresponding third shape-detected image 1530. Additionally, an overlay image 1535 superimposing the detected shape information 1245 of FIG. 12 of the shape-detected images 1520, 1525, 1530 is illustrated. The overlay image 1535 illustrates circles 1522, 1527, 1532 of the shape-detected images 1520, 1525, 1530 and their respective centers 1537.

In an example, the decision circuitry 1260 of FIG. 12 may first determine whether the single-frame approach successfully detects the presence of the sheath. If the single-frame approach finds the presence of the sheath, a transition position may be determined and the endoscope may be calibrated to a standard protrusion. However, if the single-frame approach does not find the presence of the sheath, the decision circuitry 1260 may continue to the multi-frame approach. In some examples, the multi-frame approach may have less restrictive conditions compared to the single-frame approach to function as a fallback.

The multi-frame approach may analyze the last N frames. An example illustrated in FIG. 15 shows where N=“3” but any number of frames may be analyzed. The multi-frame approach can involve aggregating the information 1230, 1235, 1240, 1245, 1250 for the last N frames and comparing the aggregated information to one or more criteria. In some examples, the one or more criteria can be the following thresholds conditions.

A first threshold condition may determine whether the total number of pixels counted aggregated from the full mask information 1230 for the last N images exceeds a first threshold value. In some implementations, the first threshold value may be, about 10% of the total number of pixels in the N images, about 15% of the total number of pixels in the N images, about 20% of the total number of pixels in the N images, about 25% of the total number of pixels in the N images, etc.

A second threshold condition may determine whether any of the pixel count in one of the inverse circular quadrants aggregated from the inverse circle mask information 1235 for the last N images exceeds a second threshold value. In some implementations, the second threshold value may be, about 2.5% of the total number of pixels in the N images, about 5% of the total number of pixels in the N images, about 7.5% of the total number of pixels in the N images, about 10% of the total number of pixels in the N images, about 15% of the total number of pixels in the N images, etc.

A third threshold condition may determine whether a range computed for the collection of centers 1537 for the N images falls short of a third threshold value (e.g., a limit value). In one example, the range may be computed as a standard deviation of the collection of centers 1537.

Various other threshold conditions (or other criteria) may be used. It is noted that one or more threshold conditions may be dropped in implementations with less strict sheath detection criteria. Conversely, one or more threshold conditions may be added in implementations with more strict sheath detection criteria.

Furthermore, any threshold condition may be combined with another threshold condition to detect the sheath. For example, the multi-frame approach may determine that the sheath is detected when (i) the first threshold condition and the third threshold condition are satisfied or when (ii) the second threshold condition and the third threshold condition are satisfied.

### Example Images

FIG. 16 is a set of images 1600 showing a detected sheath image 1605, no detection image 1610, and an insufficient detection image 1615, in accordance with some implementations. The set is for illustrative purposes and is not to be considered as limiting detection capability of the present disclosure.

The detected sheath image 1605 shows a sheath 1620, the perimeter 1625 of the sheath opening, and outside 1630 of the sheath. The detected sheath image 1605 satisfies the threshold conditions describe in relation to FIG. 10. Thus, the calibration process described in relation to FIGS. 7A and 7B may terminate retraction based on the detected sheath image 1605 and determine the transition position of the sheath relative to the scope.

The no detection image 1610 does not show any portion of the sheath. During a calibration, the scope would begin retraction and continue being retracted until the sheath is visible to the camera and the image taken by the camera exceeds a threshold subsequent to image processing. A position of the camera attached to the distal end of the scope that took the no detection image 1610 may be protruding from a distal end of the sheath, aligned with the sheath, or too slightly inside the sheath (e.g., −0.05 mm negative protrusion) to capture the perimeter of the sheath.

The insufficient detection image 1615 shows portions of the sheath at the top left and top right corners of the image. However, those portions do not satisfy the threshold conditions for sheath detection described in relation to FIG. 10. Here, the camera proximate the distal end of the scope should be retracted further within the sheath so that there would be enough of the sheath (e.g., similar amount shown in the detected sheath image 1605) for the calibration process.

### Example Graphical User Interface

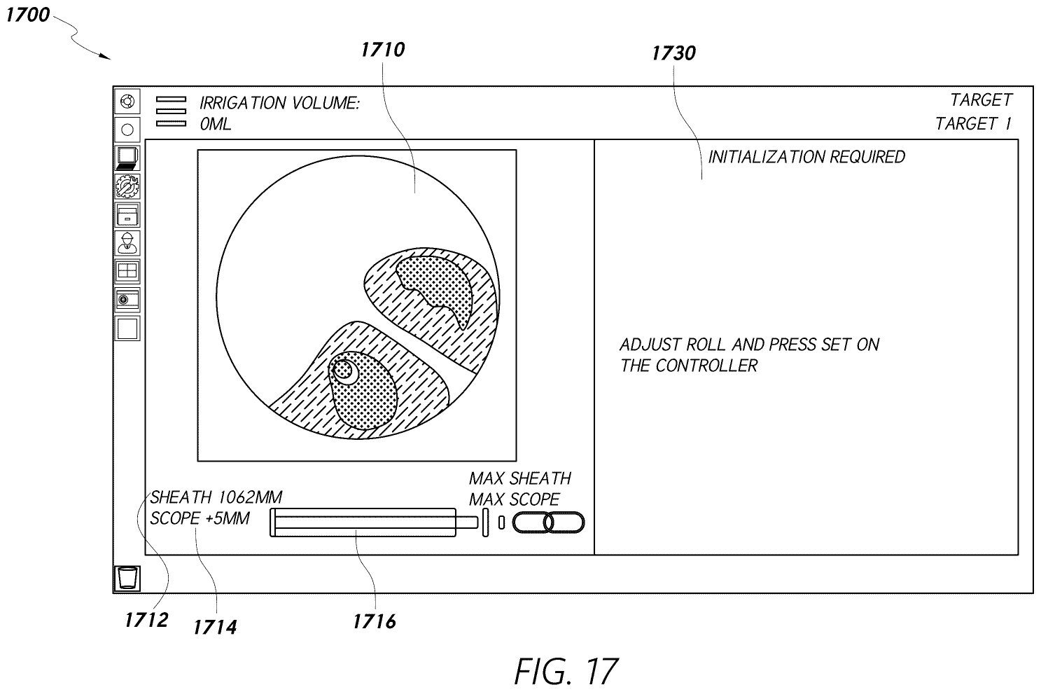

FIG. 17 is an example user interface (UI) 1700 for protrusion calibration, in accordance with some implementations. The example UI 1700 may include multiple sub-displays. In the example shown in FIG. 17, the example UI 1700 may show any or all of a current camera image 1710, a total sheath articulation 1712, and protrusion 1714 of the scope relative to the total sheath articulation 1712. The example UI 1700 further shows an illustration 1716 of the scope and sheath pair.

The right side of the example UI 1700 may show instructions 1730 based on various criteria. In various examples, the control system 50 or robotic system 10 may provide an instruction to the physician to implement protrusion calibration in the instructions 1730 area of the example UI 1700.

### Example Computer System

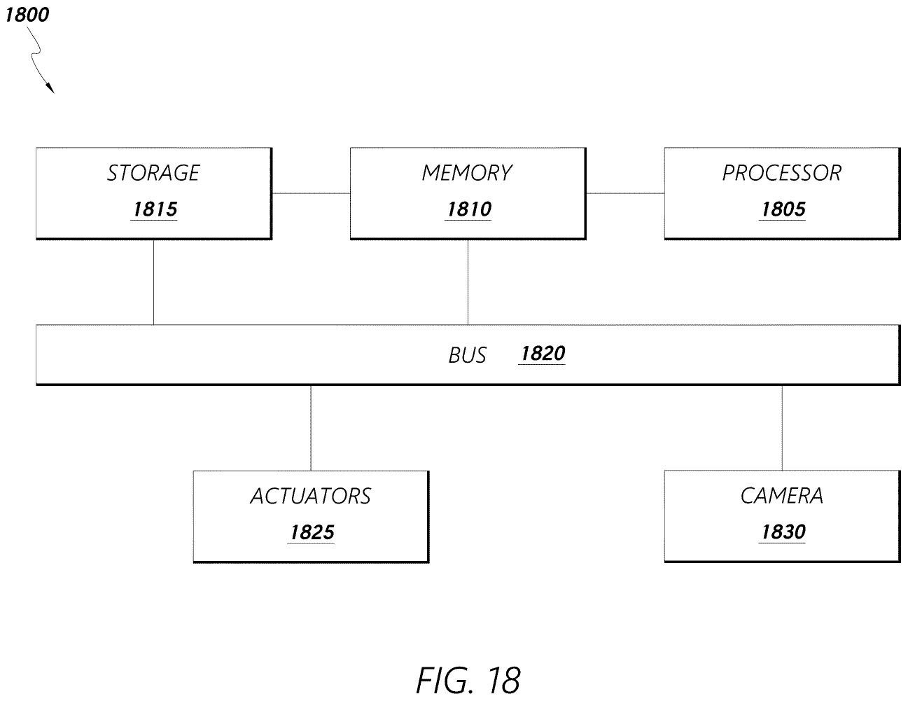

FIG. 18 is a schematic of a computer system 1800 that may be implemented by the control system 50, robotic system 10, or any other component or module in the disclosed subject matter that performs computations, stores data, processes data, and executes instructions, in accordance with some implementations. The computer system 1800 may be a single computing system, multiple networked computer systems, a co-located computing system, a cloud computing system, or the like.

The computer system 1800 may comprise a processor 1805 coupled to a memory 1810. The processor 1805 may be a single processor or multiple processors. The processor 1805 processes instructions that are passed from the memory 1810. Examples of processors 1805 are central processing units (CPUs), graphics processing units (GPUs), complex programmable logic devices (CPLDs), field programmable gate arrays (FPGAs), and application specific integrated circuits (ASICs).

The bus 1820 connects the various components of the computer system 1800 to a memory 1810. The memory 1810 receives data from the various components and transmits them according to instructions from the processor. Examples of memory 1810 include random access memory (RAM) and read only memory (ROM). The storage 1815 may store large amounts of data over a period of time. Examples of a storage 1815 include spinning disk drives and solid-state drives.

The computer system 1800 may be connected to various components of an endoscope (e.g., the endoscope 32 of FIG. 2) including a camera 1830 (the camera 48 of FIG. 2) at a distal end of a scope (e.g., the scope 30 of FIG. 2) and one or more actuators 1825 (e.g., the actuators 226 of FIG. 2) that control movement of robotic arms (e.g., the robotic arms 12), rotation of the scope and sheath (e.g., the sheath 40 of FIG. 2), and translation of the scope and sheath. The computations of the calibration process may be implemented by the computer system 1800. For instance, one or more camera images may be filtered by the computer system. The computer system 1800 may further process the filtered images to detect a circle. The computer system 1800 may further implement a detection circuitry (e.g., the decision circuitry 1260 of FIG. 13) to determine whether the sheath is detected in the camera images. Instructions to retract, extrude, or otherwise translate the scope, sheath, or other moveable components of the endoscope may be sent to the actuators 1825. For instance, instructions to retract the scope relative to the sheath during calibration may be transmitted to the actuators 1825 from the processor 1805. Further, instructions to provide the scope at a commanded protrusion relative to the sheath may be transmitted to the actuators 1825.

### Additional Embodiments

Depending on the embodiment, certain acts, events, or functions of any of the processes or algorithms described herein can be performed in a different sequence, may be added, merged, or left out altogether. In addition, various features of different disclosed embodiments can be combined to form additional embodiments, which are part of this disclosure. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A robotic system, comprising:
an instrument comprising a scope and a sheath, the sheath aligned with the scope on a coaxial axis and surrounding the scope, the scope having a sensor proximate a distal end of the scope; and
at least one computer-readable memory in communication with at least one processor, the memory having stored thereon computer-executable instructions that when executed cause the at least one processor to:
calibrate a relative position of the distal end of the scope in relation to a distal end of the sheath based at least in part on a detection of the distal end of the sheath with sensor data captured with the sensor.

2. The robotic system of claim 1, wherein the computer-executable instructions further cause the at least one processor to:
execute a movement of the scope on the coaxial axis relative to the sheath,
wherein the detection is determined during the movement.

3. The robotic system of claim 2, wherein the detection is determined during a retraction of the scope on the coaxial axis relative to the sheath.

4. The robotic system of claim 1, wherein the calibration comprises executing an extension of the scope on the coaxial axis after the detection to position the distal end of the scope at a standard protrusion in relation to the distal end of the sheath.

5. The robotic system of claim 1, wherein the detection is determined based on a transition position, the transition position representing a position of the distal end of the scope relative to the distal end of the sheath whereby the at least one processor transitions between not detecting the sheath and detecting the sheath.

6. The robotic system of claim 1, wherein the detection comprises:
filtering one or more images from the sensor that is a camera based on a color of the sheath; and
determining that a filtered portion of the one or more images satisfies a threshold condition.

7. The robotic system of claim 6, wherein determining that the filtered portion of the one or more images satisfies the threshold condition comprises analyzing a single image.

8. The robotic system of claim 6, wherein determining that the filtered portion of the one or more images satisfies the threshold condition comprises analyzing multiple images.

9. The robotic system of claim 6, wherein determining that the filtered portion of the one or more images satisfies the threshold condition comprises comparing a pixel count of filtered portion remaining after the filtering to a threshold pixel count.

10. The robotic system of claim 6, wherein determining that the filtered portion of the one or more images satisfies the threshold condition comprises:
detecting a geometrical shape in the filtered portion.

11. The robotic system of claim 10, wherein determining that the filtered portion of the one or more images satisfies the threshold further comprises:
determining a center position of the geometrical shape that is circular; and
determining that the center position is within a range of variance.

12. The robotic system of claim 1, wherein the computer-executable instructions further cause the at least one processor to:
maintain an alignment between the scope and the sheath on a coaxial axis based on the relative position.

13. A system for calibrating an endoscope, the system comprising:
a scope;
a camera proximate a distal end of the scope;
a sheath surrounding and coaxially aligned with the scope; and
at least one computer-readable memory in communication with at least one processor, the memory having stored thereon computer-executable instructions that when executed cause the at least one processor to:
determine a transition position representing a position of a distal end of the scope relative to a distal end of the sheath where the sheath becomes detectable in an image captured by the camera; and
cause a coaxial movement of the scope relative to the sheath based at least in part on the transition position and an offset.

14. The system of claim 13, wherein the first image and the second image are captured during a change in the position of the distal end of the scope relative to the distal end of the sheath.

15. The system of claim 13, wherein the determining the transition position comprises:
filtering the second image based on a color of the sheath;
determining that a filtered portion of the second image satisfies a threshold condition; and
in response to the determination that the filtered portion satisfies the threshold condition, determining that a sheath is detected.

16. The system of claim 15, wherein the determining the transition position comprises:
generating a binary image based on the filtered portion.

17. The system of claim 15, wherein the determining that the filtered portion of the second image satisfies the threshold condition comprises:
masking the filtered portion with an inverse shape mask.

18. The system of claim 17, wherein the determining that the filtered portion of the second image satisfies the threshold condition comprises:
applying the inverse shape mask to the filtered portion to generate a masked image; and
counting pixels in each quadrant of the masked image.

19. The system of claim 15, wherein the determining that the filtered portion of the second image satisfies the threshold condition comprises:
masking the filtered portion with a segmentation mask generated using a trained neural network.

20. A method for calibrating a protrusion of a scope relative to a sheath that surrounds and is coaxially aligned with the scope, the method comprising:
capturing one or more images with a camera proximate a distal end of the scope;
filtering the one or more images based on a visual property of the sheath to generate a filtered portion;
determining that the filtered portion satisfies a threshold;
determining a transition position; and
determining a target protrusion based at least in part on the transition position.
